SEQUENCE LISTING (9 sequences)

SEQ ID NO: 1 - DNA, 1680 bp, Staphylococcus epidermidis
atgagaggatcgcatcaccatcaccatcacggatccgaggagaatacagtacaagacgttaaagattcgaatatggatgatgaattatcagatagcaatgatcagtccagtaatgaagaaaagaatgatgtaatcaataatagtcagtcaataaacaccgatgatgataaccaaataaaaaaagaagaaacgaatagcaacgatgccatagaaaatcgctctaaagatataacacagtcaacaacaaatgtagatgaaaacgaagcaacatttttacaaaagacccctcaagataatactcagcttaaagaagaagtggtaaaagaaccctcatcagtcgaatcctcaaattcatcaatggatactgcccaacaaccatctcatacaacaataaatagtgaagcatctattcaaacaagtgataatgaagaaaattcccgcgtatcagattttgctaactctaaaataatagagagtaacactgaatccaataaagaagagaatactatagagcaacctaacaaagtaagagaagattcaataacaagtcaaccgtctagctataaaaatatagatgaaaaaatttcaaatcaagatgagttattaaatttaccaataaatgaatatgaaaataaggttagaccgttatctacaacatctgcccaaccatcgagtaagcgtgtaaccgtaaatcaattagcggcagaacaaggttcgaatgttaatcatttaattaaagttactgatcaaagtattactgaaggatatgatgatagtgatggtattattaaagcacatgatgctgaaaacttaatctatgatgtaacttttgaagtagatgataaggtgaaatctggtgatacgatgacagtgaatatagataagaatacagttccatcagatttaaccgatagttttgcaataccaaaaataaaagataattctggagaaatcatcgctacaggtacttatgacaacacaaataaacaaattacctacacttttacagattatgtagataaatatgaaaatattaaagcgcaccttaaattaacatcatacattgataaatcaaaggttccaaataataacactaagttagatgtagaatataagacggccctttcatcagtaaataaaacaattacggttgaatatcaaaaacctaacgaaaatcggactgctaaccttcaaagtatgttcacaaacatagatacgaaaaaccatacagttgagcaaacgatttatattaaccctcttcgttattcagccaaagaaacaaatgtaaatatttcagggaatggcgatgaaggttcaacaattatcgacgatagtacaatcattaaagtttataaggttggagataatcaaaatttaccagatagtaacagaatttatgattacagtgaatatgaagatgtcacaaatgatgattatgcccaattaggaaataataatgacgtgaatattaattttggtaatatagattcaccatatattattaaagttattagtaaatatgaccctaataaggacgattacacgacgatacagcaaactgtgacaatgcaaacgactataaatgagtatactggtgagtttagaacagcatcctatgataatacaattgctttctctacaagttcaggtcaaggacaaggtgacttgcctcctgaaaaa

SEQ ID NO: 2 - protein, 560 aa, Staphylococcus epidermidis
MRGSHHHHHHGSEENTVQDVKDSNMDDELSDSNDQSSNEEKNDVINNSQSINTDDDNQIKKEETNSNDAIENRSKDITQSTTNVDENEATFLQKTPQDNTQLKEEVVKEPSSVESSNSSMDTAQQPSHTTINSEASIQTSDNEENSRVSDFANSKIIESNTESNKEENTIEQPNKVREDSITSQPSSYKNIDEKISNQDELLNLPINEYENKVRPLSTTSAQPSSKRVTVNQLAAEQGSNVNHLIKVTDQSITEGYDDSDGIIKAHDAENLIYDVTFEVDDKVKSGDTMTVNIDKNTVPSDLTDSFAIPKIKDNSGEIIATGTYDNTNKQITYTFTDYVDKYENIKAHLKLTSYIDKSKVPNNNTKLDVEYKTALSSVNKTITVEYQKPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEK

SEQ ID NO: 3 - DNA, 1030 bp, Staphylococcus epidermidis
atgagaggatcgcatcaccatcaccatcacggatctctggttcctaggggatccgaacaaggttcgaatgttaatcatttaattaaagttactgatcaaagtattactgaaggatatgatgatagtgatggtattattaaagcacatgatgctgaaaacttaatctatgatgtaacttttgaagtagatgataaggtgaaatctggtgatacgatgacagtgaatatagataagaatacagttccatcagatttaaccgatagttttgcaataccaaaaataaaagataattctggagaaatcatcgctacaggtacttatgacaacacaaataaacaaattacctacacttttacagattatgtagataaatatgaaaatattaaagcgcaccttaaattaacatcatacattgataaatcaaaggttccaaataataacactaagttagatgtagaatataagacggccctttcatcagtaaataaaacaattacggttgaatatcaaaaacctaacgaaaatcggactgctaaccttcaaagtatgttcacaaacatagatacgaaaaaccatacagttgagcaaacgatttatattaaccctcttcgttattcagccaaagaaacaaatgtaaatatttcagggaatggcgatgaaggttcaacaattatcgacgatagtacaatcattaaagtttataaggttggagataatcaaaatttaccagatagtaacagaatttatgattacagtgaatatgaagatgtcacaaatgatgattatgcccaattaggaaataataatgacgtgaatattaattttggtaatatagattcaccatatattattaaagttattagtaaatatgaccctaataaggacgattacacgacgatacagcaaactgtgacaatgcaaacgactataaatgagtatactggtgagtttagaacagcatcctatgataatacaattgctttctctacaagttcaggtcaaggacaaggtgacttgcctcctgaaaaat

SEQ ID NO: 4 - protein, 343 aa, Staphylococcus epidermidis
MRGSHHHHHHGSLVPRGSEQGSNVNHLIKVTDQSITEGYDDSDGIIKAHDAENLIYDVTFEVDDKVKSGDTMTVNIDKNTVPSDLTDSFAIPKIKDNSGEIIATGTYDNTNKQITYTFTDYVDKYENIKAHLKLTSYIDKSKVPNNNTKLDVEYKTALSSVNKTITVEYQKPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEK

SEQ ID NO: 5 - DNA, 951 bp, Staphylococcus epidermidis
atgagaggatcgcatcaccatcaccatcacggatccgaacaaggttcgaatgttaatcatttaattaaagttactgatcaaagtattactgaaggatatgatgatagtgatggtattattaaagcacatgatgctgaaaacttaatctatgatgtaacttttgaagtagatgataaggtgaaatctggtgatacgatgacagtgaatatagataagaatacagttccatcagatttaaccgatagttttgcaataccaaaaataaaagataattctggagaaatcatcgctacaggtacttatgacaacacaaataaacaaattacctacacttttacagattatgtagataaatatgaaaatattaaagcgcaccttaaattaacatcatacattgataaatcaaaggttccaaataataacactaagttagatgtagaatataagacggccctttcatcagtaaataaaacaattacggttgaatatcaaaaacctaacgaaaatcggactgctaaccttcaaagtatgttcacaaacatagatacgaaaaaccatacagttgagcaaacgatttatattaaccctcttcgttattcagccaaagaaacaaatgtaaatatttcagggaatggcgatgaaggttcaacaattatcgacgatagtacaatcattaaagtttataaggttggagataatcaaaatttaccagatagtaacagaatttatgattacagtgaatatgaagatgtcacaaatgatgattatgcccaattaggaaataataatgacgtgaatattaattttggtaatatagattcaccatatattattaaagttattagtaaatatgaccctaataaggacgattacacgacgatacagcaaactgtgacaatgcaaacgactataaatgagtatactggtgagtttagaacagcatcctattga

SEQ ID NO: 6 - protein, 316 aa, Staphylococcus epidermidis
MRGSHHHHHHGSEQGSNVNHLIKVTDQSITEGYDDSDGIIKAHDAENLIYDVTFEVDDKVKSGDTMTVNIDKNTVPSDLTDSFAIPKIKDNSGEIIATGTYDNTNKQITYTFTDYVDKYENIKAHLKLTSYIDKSKVPNNNTKLDVEYKTALSSVNKTITVEYQKPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASY

SEQ ID NO: 7 - protein, 16 aa, Staphylococcus epidermidis
CNEEGFFSARGHRPLD

SEQ ID NO: 8 - protein, 1092 aa, Staphylococcus epidermidis
MINKKNNLLTKKKPIANKSNKYAIRKFTVGTASIVIGATLLFGLGHNEAKAEENSVQDVKDSNTDDELSDSNDQSSDEEKNDVINNNQSINTDDNNQIIKKEETNNYDGIEKRSEDRTESTTNVDENEATFLQKTPQDNTHLTEEEVKESSSVESSNSSIDTAQQPSHTTINREESVQTSDNVEDSHVSDFANSKIKESNTESGKEENTIEQPNKVKEDSTTSQPSGYTNIDEKISNQDELLNLPINEYENKARPLSTTSAQPSIKRVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEKTYKIGDYVWEDVDKDGIQNTNDNEKPLSNVLVTLTYPDGTSKSVRTDEDGKYQFDGLKNGLTYKITFETPEGYTPTLKHSGTNPALDSEGNSVWVTINGQDDMTIDSGFYQTPKYSLGNYVWYDTNKDGIQGDDEKGISGVKVTLKDENGNIISTTTTDENGKYQFDNLNSGNYIVHFDKPSGMTQTTTDSGDDDEQDADGEEVHVTITDHDDFSIDNGYYDDESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSVSDSDSDSDSDSGSDSDSDSDSDSDNDSDLGNSSDKSTKDKLPDTGANEDYGSKGTLLGTLFAGLGALLLGKRRKNRKNKN

SEQ ID NO: 9 - protein, 549 aa, Staphylococcus epidermidis
EENSVQDVKDSNTDDELSDSNDQSSDEEENDVINNNQSINSDDNNQINKKEETNNNDGIEKSSEDRTESTTNVDENEATFLQKSPQDNTHLTEEEVKEPSSVESSNSSIDTAQQPSHTTINREESVQTSDNVEDSHVSDFANSKIKESNTESGKEENTIEQPNKVKEDSTTSQPSGYTNIDEKISNQDELLNLPINEYENKARPLSTTSAQPSIKRVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEK

(19) United States
(12) Patent Application Publication
(10) Pub. No.: US 2004/0006209 A1
(43) Pub. Date: Jan. 8, 2004

(54) Monoclonal and polyclonal antibodies recognizing coagulase-negative staphylococcal proteins

(76) Inventors: Joseph M. Patti, Cumming, GA (US); Jeff T. Hutchins, Cumming, GA (US); Andrea Hall, Acworth, GA (US); Paul Domanski, Atlanta, GA (US); Pratiksha Patel, Marietta, GA (US); Magnus Hook, Houston, TX (US); Jeff Robbins, Alpharetta, GA (US); John Vernachio, Canton, GA (US); Maria Bowden, Sugarland, TX (US)

Correspondence Address: LARSON & TAYLOR, PLC, 1199 NORTH FAIRFAX STREET, SUITE 900, ALEXANDRIA, VA, 22314, US

(21) Appl. No.: 10/378,674

(22) Filed: Mar. 5, 2003

Related U.S. Application Data
(60) Provisional application No. 60/361,324, filed on Mar. 5, 2002.

(51) Int. Cl. C07K 1/00; C07K 14/00; C07K 17/00

(52) U.S. Cl. 530/350

(57) ABSTRACT

Monoclonal and polyclonal antibodies are provided which recognize and bind to the SdrG protein of S. epidermidis, and more particularly to antibodies which recognize specific domains of the SdrG protein, namely the SdrG N1N2N3 protein (amino acids 50-597), the SdrG N2N3 protein (amino acids 273-597) and a truncated version of N2N3 identified as SdrG TR2 (amino acids 273-577). The antibodies of the invention, as well as pharmaceutical compositions incorporating these antibodies, are particularly useful in treating or preventing infections caused by coagulase-negative staphylococci

### CROSS-REFERENCE TO RELATED APPLICATIONS

The present application claims the benefit of U.S. provisional application Ser. No. 60/361,324, filed Mar. 5, 2002.

### FIELD OF THE INVENTION

The present invention relates to the fields of microbiology, molecular biology, and immunology and more particularly relates to newly identified monoclonal antibodies, the use of monoclonal antibodies, as well as the production of such monoclonal antibodies and recombinant host cells transformed with the DNA encoding monoclonal antibodies to prevent, treat, or diagnose coagulase-negative staphylococcal infections in man and animals. The invention includes murine, chimeric, humanized, and human monoclonal antibodies, as well as fragments, regions and derivatives thereof. In addition, the invention relates to polyclonal antibodies generated against specific domains of the SdrG protein which are useful in treating or preventing coagulase-negative staphylococcal infections. The antibodies detailed in this invention have been generated from SdrG proteins such as SdrG N1N2N3, N2N3 and TR2, and specifically recognize SdrG, a fibrinogen binding MSCRAMM® protein expressed by coagulase-negative staphylococci such as S. epidermidis.

### BACKGROUND OF THE INVENTION

Coagulase-negative staphylococci, such as Staphylococcus epidermidis, are generally avirulent commensal organisms of the human skin and the principle etiologic agent of infections of peripheral and central venous catheters, prosthetic heart valves, artificial joints, and other prosthetic devices. S. epidermidis bacteremia has an attributable mortality rate of 10-34% and results in an excess hospital stay of 8 days, with costs for such a stay reaching $6,000.00 or more per case. Despite its importance as a nosocomial pathogen, relatively little is known about the pathogenesis of these infections or the virulence determinants of this organism. Initial localized infections of indwelling medical devices can lead to more serious invasive infections such as septicemia, osteomyelitis, and endocarditis. Vascular catheters are thought to become infected when microorganisms gain access to the device, and hence the bloodstream, by migration from the skin surface down the transcutaneous portion of the catheter. In infections associated with medical devices, plastic and metal surfaces become coated with host plasma and matrix proteins such as fibrinogen, vitronectin and fibronectin shortly after implantation.

It is now well established that the ability of coagulase-negative staphylococci to adhere to these proteins is of crucial importance for initiating infection. Bacterial or microorganism adherence is thought to be the first crucial step in the pathogenesis of a prosthetic device infection. A number of factors influence an organism's ability to adhere to prosthetic material. These include characteristics of the microorganism and the biomaterial, and the nature of the ambient milieu. The initial attraction between the organism and the host is influenced by nonspecific forces such as surface charge, polarity, Van der Waal forces and hydrophobic interactions. The critical stage of adherence involves specific interactions between MSCRAMM® proteins and immobilized host proteins.

To date, investigation concerning the adherence of coagulase negative staphylococci to biomaterials has concerned itself primarily with the role of the extracellular polysaccharide or glycocalyx, also known as slime. Despite intensive study however, the proposed role of slime in the pathogenesis of disease or even its composition remain debated. Drewry. D. T., L Gailbraith. B. I. Wilkinson, and S. G. Wilkinson. 1990. Staphylococcal Slime: A Cautionary Tale, I. Clin. MicrobioL28:1292-1296. Currently, extracellular slime is thought to play a role in the later stages of adherence and persistence of infection. It may serve as an ion exchange resin to optimize a local nutritional environment, prevent penetration of antibiotics into the macro-colony and protect bacteria from phagocytic host defense cells. Peters et al have shown by electron microscopy studies that extracellular polysaccharide appears in the later stages of attachment and is not present during the initial phase of adherence. O. Peters, R. Locci. and G. Pulverer. 1982. Adherence and Growth of Coagulase-Negative Staphylococci on Surfaces in Intravenous Catheters. I. Infect. Dis. 65146:479-482. Hogt et al demonstrated that removal of the extracellular slime layer by repeated washing does not diminish the ability of S. epidermidis to adhere to biomaterials. Hogt. A. H., I. Dankert, I. A. DeVries. and I. Feijen, 1983. Adhesion of Coagulase-Negative Staphylococci to Biomaterials. J. Gen. Microbial. 129:2959-2968.

Thus, the study of the extracellular polysaccharide or exopolysaccharide has lended little to prevention of initial adherence by the bacteria. Several other studies have identified other potential adhesins of S. epidermidis including the polysaccharide adhesion (PS/A) observed by Tojo et at. Tojo, M., N. Yamashita, D. A. Goldmann. and G. B. Pier, 1988. Isolation and Characterization of a Capsular Polysaccharide Adhesin 10 from Staphylococcus epidermidis. J. Infect Dis. 157:713-722; and the slime associated antigen at (SAA) of Christensen et al. Christensen. G. D., Barker, L. P., Manhinnes, T. P., Baddour, L. M., Simpson. W. A. Identification of an Antigenic Marker of Slime Production for Staphylococcus epidermidis. Infect Immun. 1990; 58:2906-2911.

It has been demonstrated that PS/A is a complex mixture of monosaccharides and purified PS/A blocks adherence of PS/A producing strains of S. epidermidis. In an animal model of endocarditis antibodies directed against PS/A was protective. However it is not clear whether this protective effect was specific, related to anti-adhesive effects of the antibody or due to a more generalized increase in the efficiency of opsonophagocytosis of blood borne bacteria. It has been hypothesized that each functions in different stages of the adherence process with one or more of these adhesins responsible for initial attraction while other are needed for aggregation in the macro-colonies. Despite all of these studies, factors involved in the initial adherence of S. epidermidis to biomaterials remain largely unknown and equally unknown is a practical method for preventing the first stage of infection, adherence.

Another particular problem in the medical field has been the prevention and/or treatment of coagulase negative staphylococcal infections in low birth weight infants (LBW) by passive immunization with SdrG mAb(s). LBW infants are defined as those infants born between 500-1500 g. Premature infants are born before a sufficient transfer of protective maternal antibodies through the placenta takes place. The combination of insufficient antibodies, blood losses for diagnostic purposes, less efficient phagocytosis, microbial intestinal overgrowth under selection pressure from antimicrobial treatment, and repeated invasion of otherwise sterile sites by indwelling catheters, are some of the reasons for the very high nosocomial infection rates in this vulnerable population.

It thus remains a challenge to develop compositions and methods for treating and preventing infections by coagulase-negative staphylococci, and in particular there is a great need to treat or prevent nosocomial infection in vulnerable neonates.

### SUMMARY OF THE INVENTION

Accordingly, it is an object of the present invention to provide monoclonal antibodies capable of recognizing and binding to surface proteins such as SdrG from coagulase-negative staphylococci such as S. epidermidis.

It is further an object of the present invention to develop compositions and methods which can be utilized in the treatment or prevention of nosocomial coagulase negative staphylococcal infections in low birth weight infants (LBW).

It is still further an object of the present invention to provide monoclonal antibodies which can recognize the coagulase-negative staphylococcal SdrG protein and other fibrinogen binding proteins and which can thus be used in methods and compositions to treat or prevent staphylococcal infections.

It is yet another object of the present invention to generate antibodies from the SdrG protein domains such as the N1N2N3 protein, the N2N3 protein, or a truncated version thereof, and to utilize these antibodies in methods of treating or preventing infection in humans and animals.

These and other objects are provided by virtue of the present invention which comprises the generation of monoclonal and polyclonal antibodies from the S. epidermidis SdrG protein from the SdrG regions identified as N1N2N3 (amino acids 50-597) and N2N3 (amino acids 273-597), or a truncated version thereof identified as SdrG TR2 (amino acids 273-577) which recognize and can bind to the SdrG protein and which can thus be used in compositions and method to treat or prevent infections. In addition, the present invention encompasses other uses of the antibodies of the invention including the preparation of suitable vaccines, the prevention of infection in medical instruments and prosthetic devices, and the provision of kits used to identify an infection of coagulase-negative staphylococcus.

These embodiments and other alternatives and modifications within the spirit and scope of the disclosed invention will become readily apparent to those skilled in the art from reading the present specification and/or the references cited herein, all of which are incorporated by reference.

### BRIEF DESCRIPTION OF THE DRAWING FIGURES

FIG. 1 is a graphic representation of a Biacore analysis of anti-SdrG mAbs in accordance with the invention showing inhibition with SdrG—fibrinogen binding.

FIG. 2 is a graphic representation of anti-SdrG mAbs in accordance with the invention showing inhibition of SdrG binding to β-fibrinogen peptide on the Biacore chip.

FIG. 3 is a graphic representation of inhibition of human fibrinogen binding to SdrG as shown by ELISA for monoclonal anti-SdrG antibodies in accordance with the present invention.

FIG. 4 is a graphic representation of inhibition of human fibrinogen binding of the protein identified as SEQ ID NO:9 as set forth below.

FIG. 5 is a graphic representation of the results observed in a suckling rat pup challenge model of a coagulase-negative staphylococcal (S. epidermidis) infection.

FIG. 6 is a graphic representation of the results of a central venous catheter (CVC) associated infection model of a coagulase-negative staphylococcal (S. epidermidis) infection.

### DETAILED DESCRIPTION OF THE PREFERRED EMBODIMENTS

In accordance with the present invention, there are provided antibodies which can bind to the SdrG protein of coagulase-negative bacteria such as S. epidermidis, and which have been shown to protect against S. aureus infections. The term “antibodies” as used herein includes monoclonal, polyclonal, chimeric, single chain, bispecific, simianized, and humanized or primatized antibodies as well as Fab fragments, such as those fragments which maintain the binding specificity of the antibodies to the SdrG protein, including the products of a Fab immunoglobulin expression library. Generation of any of these types of antibodies may be accomplished by suitable means well known in the art such as those described below. As explained further below, these antibodies have been generated from and can recognize and thus bind to the S. epidermidis SdrG regions identified as N1N2N3 (amino acids 50-597) and N2N3 (amino acids 273-597), as well as a truncated version of the N2N3 protein identified as TR2 (amino acids 273-597). As has been recently shown, S. epidermidis contains surface proteins structurally related to S. aureus MSCRAMM® proteins, as set forth in co-pending patent applications including pending U.S. Ser. No. 09/386,962, published as WO 00/12689, incorporated herein by reference. In addition, other information concerning staphylococcal MSCRAMM® proteins is disclosed in U.S. Ser. No. 09/386,960, published as WO 00/12132, and U.S. Ser. No. 09/386,959, published as WO 00/12131, all incorporated herein by reference. Additional information regarding MSCRAMM® proteins is disclosed in U.S. Pat. No. 6,288,214, incorporated herein by reference.

One of the proteins from S. epidermidis, namely the one identified as SdrG (serine-aspartate repeat protein G), such as disclosed in WO 00/12689, has features typical of Gram-positive bacterial proteins that are anchored to the cell wall. This protein shows significant amino acid sequence homology to ClfA and ClfB from S. aureus including an 500-amino acid-long A region, a SD dipeptide repeat region, and has features required for cell wall anchoring, including a LPXTG motif.

To date, no one has described monoclonal antibodies that specifically recognize SdrG, exhibit high affinity (>10⁸ KD), and are protective in animals models of disease. Accordingly, the present invention provides for the first time monoclonal antibodies which can specifically recognize SdrG, can bind it with high affinity, and which has been shown to be protective against Staphylococcal infection.

In accordance with the present invention, and as described further below, antibodies are generated which recognize the SdrG N1N2N3 protein at amino acids 50-597 of the S. epidermidis SdrG protein, the SdrGN2N3 protein (amino acids 273-597) and truncated version TR2 protein (amino acids 273-597), and such antibodies may be used in compositions and methods of treating or preventing coagulase-negative staphylococcal infection. In the first aspect of the invention, an isolated and/or purified version of SdrG N1N2N3, N2N3 and TR2 may be obtained in accordance with the invention in any suitable manner such as described below. The nucleic acid and amino acid sequences of these proteins are as shown below:

SdrG N1N2N3 (50-597):

| | |
|---|---|
| Nucleotide Sequence | |
| ATGAGAGGATCGCATCACCATCACCATCACGGATCCGAGGAGAATACAGTA | (SEQ ID NO:1) |
| CAAGACGTTAAAGATTCGAATATGGATGATGAATTATCAGATAGCAATGATC | |
| AGTCCAGTAATGAAGAAAAGAATGATGTAATCAATAATAGTCAGTCAATAAA | |
| CACCGATGATGATAACCAAATAAAAAAAGAAGAAACGAATAGCAACGATGCC | |
| ATAGAAAATCGCTCTAAAGATATAACACAGTCAACAACAAATGTAGATGAAA | |
| ACGAAGCAACATTTTTACAAAAGACCCCTCAAGATAATACTCAGCTTAAAGA | |
| AGAAGTGGTAAAAGAACCCTCATCAGTCGAATCCTCAAATTCATCAATGGAT | |
| ACTGCCCAACAACCATCTCATACAACAATAAATAGTGAAGCATCTATTCAAA | |
| CAAGTGATAATGAAGAAAATTCCCGCGTATCAGATTTTGCTAACTCTAAAATA | |
| ATAGAGAGTAACACTGAATCCAATAAAGAAGAGAATACTATAGAGCAACCTA | |
| ACAAAGTAAGAGAAGATTCAATAACAAGTCAACCGTCTAGCTATAAAAATAT | |
| AGATGAAAAAATTTCAAATCAAGATGAGTTATTAAATTTACCAATAAATGAAT | |
| ATGAAAATAAGGTTAGACCGTTATCTACAACATCTGCCCAACCATCGAGTAA | |
| GCGTGTAACCGTAAATCAATTAGCGGCAGAACAAGGTTCGAATGTTAATCAT | |
| TTAATTAAAGTTACTGATCAAAGTATTACTGAAGGATATGATGATAGTGATGG | |
| TATTATTAAAGCACATGATGGTGAAAACTTAATCTATGATGTAACTTTTGAAG | |
| TAGATGATAAGGTGAAATCTGGTGATACGATGACAGTGAATATAGATAAGAA | |
| TACAGTTCCATCAGATTTAACCGATAGTTTTGCAATACCAAAAATAAAAGATA | |
| ATTCTGGAGAAATCATCGCTACAGGTACTTATGACAACACAAATAAACAAAT | |
| TACCTACACTTTTACAGATTATGTAGATAAATATGAAAATATTAAAGCGCACC | |
| TTAAATTAACATCATACATTGATAAATCAAAGGTTCCAAATAATAACACTAAG | |
| TTAGATGTAGAATATAAGACGGCCCTTTCATCAGTAAATAAAACAATTACGG | |
| TTGAATATCAAAAACCTAACGAAAATCGGACTGCTAACCTTCAAAGTATGTT | |
| CACAAACATAGATACGAAAAACCATACAGTTGAGCAAACGATTTATATTAAC | |
| CCTCTTCGTTATTCAGCCAAAGAAACAAATGTAAATATTTCAGGGAATGGCG | |
| ATGAAGGTTCAACAATTATCGAGGATAGTACAATCATTAAAGTTTATAAGGTT | |
| GGAGATAATCAAAATTTACCAGATAGTAACAGAATTTATGATTACAGTGAATA | |
| TGAAGATGTCACAAATGATGAUATGCCCAATTAGGAAATAATAATGACGTG | |
| AATATTAATTTTGGTAATATAGATTCACCATATATTATTAAAGTTATTAGTAAA | |
| TATGACCCTAATAAGGACGATTACAGGACGATACAGCAAACTGTGACAATGC | |
| AAACGACTATAAATGAGTATACTGGTGAGTTTAGAACAGCAICCTATGATAA | |
| TACAATTGCTTTCTCTACAAGTTCAGGTCAAGGACAAGGTGACTTGCCTCCT | |
| GAAAAA | |
| Amino Acid Sequence | |
| MRGSHHHHHHGSEENTVQDVKDSNMDDELSDSNDQSSNEEKNDVINNSQSIN | (SEQ ID NO:2) |
| TDDDNQIKKEETNSNDAIENRSKDITQSTTNVDENEATFLQKTPQDNTQLKEEV | |
| VKEPSSVESSNSSMDTAQQPSHTTINSEASIQTSDNEENSRVSDFANSKIIESNT | |
| ESNKEENTIEQPNKVREDSITSQPSSYKNIDEKISNQDELLNLPINEYENKVRPLS | |
| TTSAQPSSKRVTVNQLAAEQGSNVNHLIKVIDQSITEGYDDSDGIIKAHDAENLI | |
| YDVTFEVDDKVKSGDTMTVNIDKNTVPSDLTDSFAIPKIKDNSGEIIATGTYDNTN | |
| KQITYTFTDYVDKYENIKAHLKLTSYIDKSKVPNNNTKLDVEYKTALSSVNKTITV | |
| EYQKPNENRTANLQSMFTNIDTKNHIVEQTIYINPLRYSAKETNVNISGNGDEG | |
| STIIDDSTIIKVYKVGDNQNLPDSNRIYDYSEYEDVINDDYAQLGNNNDVNINFG | |
| NIDSPYIIKVISKYDPNKDDYTTIQQTVTMQTTINEYTGEFRTASYDNTIAFSTSSG | |
| QGQGDLPPEK | |

SdrG N2N3 (273-597):

| | |
|---|---|
| Nucleotide Sequence: | |
| ATGAGAGGATCGCATCACCATCACCATCACGGATCTCTGGTTCCTAGGGGA | (SEQ ID NO:3) |
| TCCGAACAAGGTTCGAATGTTAATCATTTAATTAAAGTTACTGATCAAAGTAT | |
| TACTGAAGGATATGATGATAGTGATGGTATTATTAAAGCACATGATGCTGAA | |
| AACTTAATCTATGATGTAACTTTTGAAGTAGATGATAAGGTGAAATCTGGTG | |
| ATACGATGACAGTGAATATAGATAAGAATACAGTTCCATCAGATTTAACCGA | |
| TAGTTTTGCAATACCAAAAATAAAAGATAATTCTGGAGAAATCATCGCTACAG | |
| GTACTTATGAGAACACAAATAAACAAATTACCTACACTTTTACAGATTATGTA | |
| GATAAATATGAAAATATTAAAGCGCACCTTAAATTAAGATCATACATTGATAA | |
| ATCAAAGGTTCCAAATAATAACACTAAGTTAGATGTAGAAIATAAGACGGCC | |
| CTTTCATCAGTAAATAAAACAATTACGGTTGAATATCAAAAACGTAACGAAAA | |
| TCGGACTGCTAACCTTCAAAGTATGTTGACAAACATAGATACGAAAAACCAT | |
| ACAGTTGAGCAAACGATTTATATTAACCCTCTTCGTTATTCAGCCAAAGAAA | |
| CAAATGTAAATATTTCAGGGAATGGCGATGAAGGTTCAACAATTATCGACGA | |
| TAGTACAATCATTAAAGTTTATAAGGTTGGAGATAATCAAAATTTACCAGATA | |
| GTAACAGAATTTATGATTACAGTGAATATGAAGATGTCACAAATGATGATTAT | |
| GCCCAATTAGGAAATAATAATGACGTGAATATTAATTTTGGTAATATAGATTC | |
| ACCATATATTATTAAAGTTATTAGTAAATATGACCCTAATAAGGAGGATTACA | |
| CGACGATACAGCAAACTGTGACAATGCAAACGACTATAAATGAGTATACTGG | |
| TGAGTTTAGAACAGCATCCTATGATAATACAATTGCTTTCTCTACAAGTTCAG | |
| GTCAAGGACAAGGTGACTTGCCTCCTGAAAAAT | |
| Amino Acid Sequence | |
| MRGSHHHHHHGSLVPRGSEQGSNVNHLIKVTDQSITEGYDDSDGIIKAHDAENL | (SEQ ID NO:4) |
| IYDVTFEVDDKVKSGDTMIVNIDKNTVPSDLTDSFAIPKIKDNSGEIIATGTYDNT | |
| NKQITYTFTDYVDKYENIKAHLKLTSYIDKSKVPNNNTKLDVEYKTALSSVNKTIT | |
| VEYQKPNENRTANLQSMFTNIDTKNHTVEQIIYINPLRYSAKETNVNISGNGDE | |
| GSTIIDDSTIIKVYKVGDNQNLPDSNRIYDYSEYEDVINDDYAQLGNNNDVNINF | |
| GNIDSPYIIKVISKYDPNKDDYTTIQQTVTMQTTINEYTGEFRTASYDNTIAFSTSS | |
| GQGQGDLPPEK | |

SdrG TR2 (273-577):

| | |
|---|---|
| Nucleotide Sequence | |
| ATGAGAGGATCGCATCACCATCACCATCACGGATCCGAACAAGGTTCGAAT | (SEQ ID NO:5) |
| GTTAATCATTTAATTAAAGTTACTGATCAAAGTATTACTGAAGGATATGATGA | |
| TAGTGATGGTATTATTAAAGCACATGATGCTGAAAACTTAATCTATGATGTAA | |
| CTTTTGAAGTAGATGATAAGGTGAAATCTGGTGATACGATGACAGTGAATAT | |
| AGATAAGAATACAGTTCCATCAGATTTAACCGATAGTTTTGCAATACCAAAAA | |
| TAAAAGATAATTCTGGAGAAATCATCGCTACAGGTACTTATGACAACACAAA | |
| TAAACAAATTACCTACACTTTTACAGATTATGTAGATAAATATGAAAATATTAA | |
| AGCGCACCTTAAATTAACATCATACAHGATAAATCAAAGGTTCCAAATAATA | |
| ACACTAAGTTAGATGTAGAATATAAGACGGCCCTTTGATCAGTAAATAAAAC | |
| AATTACGGTTGAATATCAAAAACCTAACGAAAATCGGACTGCTAACCTTCAA | |
| AGTATGTTCACAAACATAGATACGAAAAACCATACAGTTGAGCAAACGATTT | |
| ATATTAACCCTCTTCGTTATTCAGCCAAAGAAACAAATGTAAATATTTCAGGG | |
| AATGGCGATGAAGGTTCAACAATTATCGACGATAGTACAATCATTAAAGTTT | |
| ATAAGGTTGGAGATAATCAAAATTTACCAGATAGTAACAGAATTTATGATTAC | |
| AGTGAATATGAAGATGTCACAAATGATGATTATGCCCAATTAGGAAATAATA | |
| ATGACGTGAATATTAATTTTGGTAATATAGATTCACCATATATTATTAAAGTTA | |
| TTAGTAAATATGACCCTAATAAGGACGATTACACGAGGATACAGCAAACTGT | |
| GACAATGCAAACGACTATAAATGAGTATACTGGTGAGTTTAGAACAGCATCC | |
| TATTGA | |
| Amino Acid Sequence | |
| MRGSHHHHHHGSEQGSNVNHLIKVTDQSITEGYDDSDGIIKAHDAENLIYDVTF | (SEQ ID NO:6) |
| EVDDKVKSGDTMTVNIDKNTVPSDLTDSFAIPKIKDNSGEIIATGTYDNTNKQITY | |
| TFTDYVDKYENIKAHLKLTSYIDKSKVPNNNTKLDVEYKTALSSVNKTITVEYQKP | |
| NENRTANLQSMFTNIDTKNHTVEQTIYINPLRYSAKETNVNISGNGDEGSTIIDDS | |
| TIIKVYKVGDNQNLPDSNRIYDYSEYEDVTNDDYAQLGNNNDVNINFGNIDSPYII | |
| KVISKYDPNKDDYTTIQQTVTMQTTINEYTGEFRTASY | |

Accordingly, the present invention encompasses isolated proteins as described above which have sequences such as SEQ ID NO:2, SEQ ID NO:4 or SEQ ID NO:6, as well as isolated proteins encoded by nucleic acid sequences SEQ ID NO:1, SEQ ID NO:3 or SEQ ID NO:5, or degenerates thereof. In addition, as described further below, the invention encompasses raising antibodies from these proteins and eliciting a immune response in humans or animals by administration of an immunogenic amount of the proteins.

As set forth in more detail below, the monoclonal and polyclonal antibodies of the invention may be prepared in a number of suitable ways that would be well known in the art. For example, monoclonal antibodies can be prepared using the well-established Kohler and Milstein method commonly used to generate monoclonal antibodies. In one such suitable method, mice may be injected intraperitoneally for a prolonged period with a purified recombinant protein such as the SdrG N1N2N3 or SdrGN2N3 domain or its truncated version TR2 referred to above, followed by a test of blood obtained from the immunized mice to determine reactivity to the purified protein or fragment. Following identification of mice reactive to the tested protein, lymphocytes isolated from mouse spleens are fused to mouse myeloma cells to produce hybridomas positive for the antibodies against these proteins which are then isolated and cultured, following by purification and isotyping.

As described, for example, in J. Biol. Chem. 1999, 274, 26939-26945 (incorporated herein by reference), one such suitable means for obtaining gene fragments in accordance with the invention, e.g., those corresponding to the SdrG N1N2N3 protein (aa 50-597), SdrG N2N3 protein (aa 273-597) or its truncated version TR2 (aa 273-577) is to use a process wherein they are amplified by using PCR, such as through subcloning using E. coli expression vector pQE-30 and transformation using E. coli strain JM101.

In a specific example, the proteins of the invention were obtained in a PCR process wherein SdrGN1N2N3 (representing AA 50-597) or SdrGN2N3 (representing AA 273-597) or its truncated version TR2 (AA 273-577) was amplified from S. epidermidis K28 genomic DNA (from sequences described above) and subcloned into the E. coli expression vector PQE-30 (Qiagen), which allows for the expression of a recombinant fusion protein containing six histidine residues. This vector was subsequently transformed into the E. coli strain ATCC 55151, grown in a 15-liter fermentor to an optical density (OD₆₀₀) of 0.7 and induced with 0.2 mM isopropyl-1-beta-D galactoside (IPTG) for 4 hours. The cells were harvested using an AG Technologies hollow-fiber assembly (pore size of 0.45 μm) and the cell paste frozen at −800 C. Cells were lysed in 1×PBS (10 mL of buffer/1 g of cell paste) using 2 passes through the French Press @ 1100 psi. Lysed cells were spun down at 17,000 rpm for 30 minutes to remove cell debris. Supernatant was passed over a 5-mL HiTrap Chelating (Pharmacia) column charged with 0.1M NiCl₂. After loading, the column was washed with 5 column volumes of 10 mM Tris, pH 8.0, 100 mM NaCl (Buffer A). Protein was eluted using a 0-100% gradient of 10 mM Tris, pH 8.0, 100 mM NaCl 200 mM imidazole (Buffer B) over 30 column volumes. SdrGN1N2N3, SdrGN2N3 or TR2 eluted at ˜13% Buffer B (˜26 mM imidazole). Absorbance at 280 nm was monitored. Fractions containing SdrGN1N2N3, SdrGN2N3 or TR2 were dialyzed in 1×PBS.

The protein was then put through an endotoxin removal protocol. Buffers used during this protocol were made endotoxin free by passing over a 5-mL Mono-Q sepharose (Pharmacia) column. Protein was divided evenly between 4×15 mL tubes. The volume of each tube was brought to 9 mL with Buffer A. 1 mL of 10% Triton X-114 was added to each tube and incubated with rotation for 1 hour at 4° C. Tubes were placed in a 37° C. water bath to separate phases. Tubes were spun down at 2,000 rpm for 10 minutes and the upper aqueous phase from each tube was collected and the detergent extraction repeated. Aqueous phases from the 2nd extraction were combined and passed over a 5-mL IDA chelating (Sigma) column, charged with 0.1M NiCl₂ to remove remaining detergent. The column was washed with 9 column volumes of Buffer A before the protein was eluted with 3 column volumes of Buffer B. The eluant was passed over a 5-mL Detoxigel (Sigma) column and the flow-through collected and reapplied to the column. The flow-through from the second pass was collected and dialyzed in 1×PBS. The purified product was analyzed for concentration, purity and endotoxin level before administration into the mice.

As indicated above, generation of the monoclonal antibodies in accordance with the invention may proceed using any of a number of conventional methods well known in the art such as the general Kohler and Milstein technique conventionally used in this field. In one specific example for preparing the monoclonal antibodies of the invention, E coli expressed and purified SdrG (N1N2N3, N2N3 or TR2) protein can be used to generate a panel of murine monoclonal antibodies. Briefly, a group of Balb/C or SJL mice received a series of subcutaneous immunizations of 1-10 mg of protein in solution or mixed with adjuvant. At the time of sacrifice (RIMMS) or seven days after a boost (conventional) serum was collected and titered in ELISA assays against MSCRAMMs or on whole cells (S. epidermidis). Three days after the final boost, the spleens or lymph nodes were removed, teased into a single cell suspension and the lymphocytes harvested. The lymphocytes were then fused to a P3X63Ag8.653 myeloma cell line (ATCC #CRL-1580). Cell fusion, subsequent plating and feeding were performed according to the Production of Monoclonal Antibodies protocol from Current Protocols in Immunology (Chapter 2, Unit 2.).

Any clones that were generated from the fusion were then screened for specific anti-SdrG antibody production using a standard ELISA assay. Positive clones were expanded and tested further for activity in a whole bacterial cell binding assay by flow cytometry and SdrG binding/inhibition of fibrinogen-Clf40 binding by Biacore analysis. Throughout the analysis, the flow rate remained constant at 10 ml/min. Prior to the SdrGN1N2N3, SdrGN2N3 or TR2 injection, test antibody was adsorbed to the chip via RAM-Fc binding. At time 0, SdrG (N2N3, TR2 or N1N2N3) at a concentration of 30 mg/ml was injected over the chip for 3 min followed by 2 minutes of dissociation. This phase of the analysis measured the relative association and disassociation kinetics of the Mab/SdrG interaction. In the second phase of the analysis, the ability of the Mab bound SdrG to interact and bind fibrinogen was measured. Fibrinogen at a concentration of 100 mg/ml was injected over the chip and after 3 minutes a report point is taken.

Following the generation of monoclonal antibodies as referred to above, these antibodies were tested for their ability to bind to whole bacteria. In these tests, bacterial samples (HB, 9142 or SdrG/lactococcus) were collected, washed and incubated with Mab or PBS alone (control) at a concentration of 2 mg/ml after blocking with rabbit IgG (50 mg/ml). Following incubation with antibody, bacterial cells were incubated with Goat-F_{(ab′)2}-Anti-Mouse-F_{(ab′)2}-FITC which served as the detection antibody. After antibody labeling, bacterial cells were aspirated through the FACScaliber flow cytometer to analyze fluorescence emission (excitation: 488, emission: 570). For each bacterial strain, 10,000 events were collected and measured.

From these tests, it was shown that SdrG positive hybridomas were generated in a frequency of 0.6-10% of the growth positive wells. A few of the SdrG ELISA positive hybridomas were also positive by Biacore analysis and whole cell bacterial binding by flow cytometry. Limited analysis demonstrated that Biacore negative, SdrG ELISA positive clones were consistently negative in the whole cell binding flow cytometry assay. From this analysis, a very small subpopulation of growth positive hybridoma wells that were SdrG ELISA positive, SdrG Biacore positive and flow cytometry positive on Lactococcus/SdrG were single cell cloned and characterized as candidates for potential efficacy against S. epidermidis infection models. These tests showed that monoclonal antibodies generated in accordance with the invention were effective in inhibiting or preventing infection by S. epidermidis and can thus be used in many therapeutic and other useful applications as set forth further below.

In addition to monoclonal antibodies, the present invention also contemplates generating polyclonal antibodies from the SdrG proteins as set forth above, as well as other proteins that will generate antibodies that can recognize SdrG proteins such as those described herein. Such polyclonal antibodies may be generated in any of a number of suitable ways well known in the art, such as the introduction of a purified SdrG protein such as those described herein into a suitable animal host, followed by isolation and purification of the generated antibodies produced in the host animal. In general, while it is preferred to use isolated and/or purified recombinant forms of the proteins to generate antibodies in accordance with the invention, antibodies may be generated as well from natural isolated and/or purified forms of these proteins.

In accordance with the invention, antibodies are thus produced which are generated from SdrG proteins N1N2N3, N2N3, and TR2, and such antibodies are capable of recognizing and binding SdrG proteins as well as other fibrinogen binding proteins from S. epidermidis including the proteins described further below. The isolated antibodies and proteins of the invention can also be utilized in many therapeutic applications, and such applications are described in more detail below.

Vaccines Humanized Antibodies and Adjuvants

The isolated antibodies of the present invention, or the isolated proteins as described above, may also be utilized in the development of vaccines for active and passive immunization against bacterial infections, as described further below. Further, when administered as pharmaceutical composition to a wound or used to coat medical devices or polymeric biomaterials in vitro and in vivo, the antibodies of the present invention, may be useful in those cases where there is a previous infection because of the ability of these antibodies to further restrict and inhibit bacterial binding to collagen and thus limit the extent and spread of the infection.

In addition, the antibody may be modified as necessary so that, in certain instances, it is less immunogenic in the patient to whom it is administered. For example, if the patient is a human, the antibody may be “humanized” by transplanting the complimentarity determining regions of the hybridoma-derived antibody into a human monoclonal antibody as described, e.g., by Jones et al., Nature 321:522-525 (1986) or Tempest et al. Biotechnology 9:266-273 (1991) or “veneered” by changing the surface exposed murine framework residues in the immunoglobulin variable regions to mimic a homologous human framework counterpart as described, e.g., by Padlan, Molecular 1 mm. 28:489-498 (1991), these references incorporated herein by reference. Even further, when so desired, the monoclonal antibodies of the present invention may be administered in conjunction with a suitable antibiotic to further enhance the ability of the present compositions to fight bacterial infections.

In a preferred embodiment, the antibodies may also be used as a passive vaccine which will be useful in providing suitable antibodies to treat or prevent a bacterial infection. As would be recognized by one skilled in this art, a vaccine may be packaged for administration in a number of suitable ways, such as by parenteral (i.e., intramuscular, intradermal or subcutaneous) administration or nasopharyngeal (i.e., intranasal) administration. One such mode is where the vaccine is injected intramuscularly, e.g., into the deltoid muscle, however, the particular mode of administration will depend on the nature of the bacterial infection to be dealt with and the condition of the patient. The vaccine is preferably combined with a pharmaceutically acceptable carrier to facilitate administration, and the carrier is usually water or a buffered saline, with or without a preservative. The vaccine may be lyophilized for resuspension at the time of administration or in solution.

The preferred dose for administration of an antibody composition in accordance with the present invention is that amount will be effective in preventing of treating a bacterial infection, and one would readily recognize that this amount will vary greatly depending on the nature of the infection and the condition of a patient. An “effective amount” of antibody or pharmaceutical agent to be used in accordance with the invention is intended to mean a nontoxic but sufficient amount of the agent, such that the desired prophylactic or therapeutic effect is produced. Accordingly, the exact amount of the antibody or a particular agent that is required will vary from subject to subject, depending on the species, age, and general condition of the subject, the severity of the condition being treated, the particular carrier or adjuvant being used and its mode of administration, and the like. Accordingly, the “effective amount” of any particular antibody composition will vary based on the particular circumstances, and an appropriate effective amount may be determined in each case of application by one of ordinary skill in the art using only routine experimentation. The dose should be adjusted to suit the individual to whom the composition is administered and will vary with age, weight and metabolism of the individual. The compositions may additionally contain stabilizers or pharmaceutically acceptable preservatives, such as thimerosal (ethyl(2-mercaptobenzoate-S)mercury sodium salt) (Sigma Chemical Company, St. Louis, Mo.).

In addition, an active vaccine in accordance with the invention is provided wherein an immunogenic amount of an isolated protein as described above is administered to a human or animal patient in need of such a vaccine. The vaccine may also comprise a suitable, pharmaceutically acceptable vehicle, excipient or carrier such as described above. As indicated above, an “immunogenic amount” of the antigen to be used in accordance with the invention is intended to mean a nontoxic but sufficient amount of the agent, such that an immunogenic response will be elicited in the host so that the desired prophylactic or therapeutic effect is produced. Accordingly, the exact amount of the antigen that is required will vary from subject to subject, depending on the species, age, and general condition of the subject, the severity of the condition being treated, the particular carrier or adjuvant being used and its mode of administration, and the like. Similarly, the “immunogenic amount” of any such antigenic vaccine composition will vary based on the particular circumstances, and an appropriate immunogenic amount may be determined in each case of application by one of ordinary skill in the art using only routine experimentation. The dose should be adjusted to suit the individual to whom the composition is administered and will vary with age, weight and metabolism of the individual.

In addition, the antibody compositions of the present invention and the vaccines as described above may also be administered with a suitable adjuvant in an amount effective to enhance the immunogenic response against the conjugate. For example, suitable adjuvants may include alum (aluminum phosphate or aluminum hydroxide), which is used widely in humans, and other adjuvants such as saponin and its purified component Quil A, Freund's complete adjuvant, and other adjuvants used in research and veterinary applications. Still other chemically defined preparations such as muramyl dipeptide, monophosphoryl lipid A, phospholipid conjugates such as those described by Goodman-Snitkoff et al. J. Immunol 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome lipid vesicles (Micro Vescular Systems, Inc., Nashua, N.H.) may also be useful.

Pharmaceutical Compositions

As would be recognized by one skilled in the art, the antibodies of the present invention may also be formed into suitable pharmaceutical compositions for administration to a human or animal patient in order to treat or prevent an infection caused by coagulase-negative staphylococcal bacteria. Pharmaceutical compositions containing the antibodies of the present invention as defined and described above may be formulated in combination with any suitable pharmaceutical vehicle, excipient or carrier that would commonly be used in this art, including such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art. Suitable methods of administration of any pharmaceutical composition disclosed in this application include, but are not limited to, topical, oral, anal, vaginal, intravenous, intraperitoneal, intramuscular, subcutaneous, intranasal and intradermal administration.

For topical administration, the composition is formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

Additional forms of antibody compositions, and other information concerning compositions, methods and applications with regard to other MSCRAMM® proteins and MSCRAMM® peptides will generally also be applicable to the present invention involving monoclonal antibodies and are disclosed, for example, in U.S. Pat. No. 6,288,214 (Hook et al.), incorporated herein by reference.

The antibody compositions of the present invention which are generated in particular against the SdrG proteins as set forth above may also be administered with a suitable adjuvant in an amount effective to enhance the immunogenic response against the conjugate. For example, suitable adjuvants may include alum (aluminum phosphate or aluminum hydroxide), which is used widely in humans, and other adjuvants such as saponin and its purified component Quil A, Freund's complete adjuvant, RIBI adjuvant, and other adjuvants used in research and veterinary applications. Still other chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome™ lipid vesicles (Micro Vescular Systems, Inc., Nashua, N.H.) may also be useful.

In any event, the antibody compositions of the present invention will thus be useful for interfering with, modulating, inhibiting binding interactions involving fibrinogen binding proteins as would take place with bacteria from coagulase-negative staphylococci. Accordingly, the present invention will have particular applicability in developing compositions and methods of preventing or treating coagulase-negative staphylococcal infection, and in inhibiting binding of staphylococcal bacteria to host tissue and/or cells.

Methods:

Treating or Protecting Against Infections

In accordance with the present invention, methods are provided for preventing or treating a coagulase-negative staphylococcal infection which comprise administering an effective amount of the antibodies as described above to a human or animal patient in need of such treatment in amounts effective to treat or prevent the infection. In addition, antibodies in accordance with the invention will be particularly useful in impairing the binding of a variety of bacteria to fibrinogen, and have thus proved effective in treating or preventing infection from bacteria such as coagulase-negative staphylococci by inhibiting said binding.

Accordingly, in accordance with the invention, administration of an effective amount of the antibodies of the present invention in any of the conventional ways described above (e.g., topical, parenteral, intramuscular, etc.), and will thus provide an extremely useful method of treating or preventing coagulase-negative staphylococcal infections in human or animal patients. As indicated above, by effective amount is meant that level of use, such as of an antibody titer, that will be sufficient to either prevent adherence of the bacteria, to inhibit binding of bacteria to host cells and thus be useful in the treatment or prevention of a bacterial infection. As would be recognized by one of ordinary skill in this art, the level of antibody titer needed to be effective in treating or preventing infections will vary depending on the nature and condition of the patient, and/or the severity of the pre-existing infection.

Eliciting an Immune Response

In accordance with the present invention, a method is provided for eliciting an immunogenic reaction in a human or animal comprising administering to the human or animal an immunologically effective amount of an isolated protein as described above, such as SdrG N1N2N3, SdrG N2N3 or SdrG TR2. As indicated above, an “immunogenic amount” of the antigen to be used in accordance with the invention to obtain an immunogenic reaction is intended to mean a nontoxic but sufficient amount of the agent, such that an immunogenic response will be elicited in the host so that the desired prophylactic or therapeutic effect is produced. Accordingly, the exact amount of the isolated protein that is required to elicit such a response will vary from subject to subject, depending on the species, age, and general condition of the subject, the severity of the condition being treated, the particular carrier or adjuvant being used and its mode of administration, and the like. The invention also contemplates methods of generating antibodies which recognize the SdrG proteins as described above, and suitable methods of generating monoclonal and polyclonal antibodies are described in more detail above.

Coating devices

In accordance with the invention, the antibodies and compositions as described above may also be utilized to treat or protect against outbreaks of coagulase-staphylococcal infections on medical devices and other implanted materials such as prosthetic devices. Medical devices or polymeric biomaterials that may be advantageously coated with the antibodies and/or compositions described herein include, but are not limited to, staples, sutures, replacement heart valves, cardiac assist devices, hard and soft contact lenses, intraocular lens implants (anterior chamber or posterior chamber), other implants such as corneal inlays, kerato-prostheses, vascular stents, epikeratophalia devices, glaucoma shunts, retinal staples, scleral buckles, dental prostheses, thyroplastic devices, laryngoplastic devices, vascular grafts, soft and hard tissue prostheses including, but not limited to, pumps, electrical devices including stimulators and recorders, auditory prostheses, pacemakers, artificial larynx, dental implants, mammary implants, penile implants, cranio/facial tendons, artificial joints, tendons, ligaments, menisci, and disks, artificial bones, artificial organs including artificial pancreas, artificial hearts, artificial limbs, and heart valves; stents, wires, guide wires, intravenous and central venous catheters, laser and balloon angioplasty devices, vascular and heart devices (tubes, catheters, balloons), ventricular assists, blood dialysis components, blood oxygenators, urethral/ureteral/urinary devices (Foley catheters, stents, tubes and balloons), airway catheters (endotracheal and tracheostomy tubes and cuffs), enteral feeding tubes (including nasogastric, intragastric and jejunal tubes), wound drainage tubes, tubes used to drain the body cavities such as the pleural, peritoneal, cranial, and pericardial cavities, blood bags, test tubes, blood collection tubes, vacutainers, syringes, needles, pipettes, pipette tips, and blood tubing.

It will be understood by those skilled in the art that the term “coated” or “coating”, as used herein, means to apply the antibody or composition as defined above to a surface of the device, preferably an outer surface that would be exposed to a bacterial infection. The surface of the device need not be entirely covered by the protein, antibody or active fragment.

As indicated above, the antibodies of the present invention, or active portions or fragments thereof, are particularly useful for interfering with the initial physical interaction between a bacterial pathogen responsible for infection and a mammalian host, such as the adhesion of the bacteria to mammalian extracellular matrix proteins such as fibrinogen, and this interference with the physical interaction may be useful both in treating patients and in preventing or reducing bacteria infection on in-dwelling medical devices to make them safer for use.

Kits

In accordance with the present invention, the antibodies of the invention as set forth above may be used in kits to diagnose an infection by coagulase-negative staphylococci such as S. epidermidis. Such diagnostic kits are well known in the art and will generally be prepared so as to be suitable for determining the presence of bacteria or proteins that will bind to the antibodies of the invention. These diagnostic kits will generally include the antibodies of the invention along with suitable means for detecting binding by that antibody such as would be readily understood by one skilled in this art. For example, the means for detecting binding of the antibody may comprise a detectable label that is linked to said antibody. These kits can then be used in diagnostic methods to detect the presence of a coagulase-negative staphylococcal infection wherein one obtains a sample suspected of being infected by one or more coagulase-negative staphylococcal bacteria, such as a sample taken from an individual, for example, from one's blood, saliva, tissues, bone, muscle, cartilage, or skin, introduces to the sample one or more of the antibodies as set forth herein, and then determines if the antibodies bind to the sample which would indicated the presence of such bacteria in the sample.

In short, the antibodies of the present invention as described above can be extremely useful in inhibiting fibrinogen binding and in treating or preventing the infection of humans, animals, or medical devices and prosthesis that can be caused by coagulase-negative staphylococcal bacteria. In particular, the present invention will be of importance in the treatment or prevention of nosocomial coagulase negative staphylococcal infections in low birth weight infants (LBW).

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Expression and Purification of SdrG Proteins

In accordance with the present invention, proteins obtained from the relevant domains of the SdrG protein were cloned, expressed recombinantly and isolated and/or purified. The SdrG N1N2N3 protein (50-597) represents the putative A domain of the SdrG gene. SdrG N2N3 protein (273-597) represents the sub-domain required for human fibrinogen binding. SdrG TR2 protein (273-577) represents the sub-domain required for human fibrinogen binding with the C-terminal portion removed that stabilizes fibrinogen binding. The nucleotide and amino acid sequences for these proteins are set forth below:

16/30 SdrG N1N2N3 (50-597):

| | |
|---|---|
| Nucleotide Sequence | |
| ATGAGAGGATCGCATCACCATCACCATCACGGATCCGAGGAGAATACAGTA | (SEQ ID NO:1) |
| CAAGACGTTAAAGATTCGAATATGGATGATGAATTATCAGATAGCAATGATC | |
| AGTCCAGTAATGAAGAAAAGAATGATGTAATCAATAATAGTCAGTCAATAAA | |
| CACCGATGATGATAACCAAATAAAAAAAGAAGAAACGAATAGCAACGATGCC | |
| ATAGAAAATCGCTCTAAAGATATAACACAGTCAACAACAAATGTAGATGAAA | |
| ACGAAGCAACATTTTTACAAAAGACCCCTCAAGATAATACTCAGCTTAAAGA | |
| AGAAGTGGTAAAAGAACCCTCATCAGTCGAATCCTCAAATTCATCAATGGAT | |
| ACTGCCCAACAACCATCTCATACAACAATAAATAGTGAAGCATCTATTCAAA | |
| CAAGTGATAATGAAGAAAATTCCCGCGTATCAGATTTTGCTAACTCTAAAATA | |
| ATAGAGAGTAACACTGAATCCAATAAAGAAGAGAATACTATAGAGCAACCTA | |
| ACAAAGTAAGAGAAGATTCAATAACAAGTCAACCGTCTAGCTATAAAAATAT | |
| AGATGAAAAAATTTCAAATCAAGATGAGTTATTAAATTTACCAATAAATGAAT | |
| ATGAAAATAAGGTTAGACCGHATCTACAACATCTGCCCAACCATCGAGTAA | |
| GCGTGTAACCGTAAATCAATTAGCGGCAGAACAAGGTTCGAATGTTAATCAT | |
| TTAATTAAAGTTACTGATCAAAGTATTACTGAAGGATATGATGATAGTGATGG | |
| TATTATTAAAGCACATGATGCTGAAAACTTAATCTATGATGTAACTTTTGAAG | |
| TAGATGATAAGGTGAAATCTGGTGATACGATGACAGTGAATATAGATAAGAA | |
| TACAGTTCCATCAGATTTAACCGATAGTTTTGCAATACCAAAAATAAAAGATA | |
| ATTCTGGAGAAATCATCGCTACAGGTACTTATGACAACACAAATAAACAAAT | |
| TACCTACACTTTTACAGATTATGTAGATAAATATGAAAATATTAAAGCGCACC | |
| TTAAATTAACATCATACATTGATAAATCAAAGGTTCCAAATAATAACACTAAG | |
| TIAGATGTAGAATATAAGACGGCCCTTTCATCAGTAAATAAAACAATTACGG | |
| TTGAATATCAAAAACCTAACGAAAATCGGACTGCTAACCTTCAAAGTATGTT | |
| CACAAACATAGATACGAAAAACCATACAGTTGAGCAAACGATTTATATTAAC | |
| CCTCTTCGTTATTCAGCCAAAGAAACAAATGTAAATATTTCAGGGAATGGCG | |
| ATGAAGGTTCAACAATTATCGAGGATAGTACAATCATTAAAGTTTATAAGGTT | |
| GGAGATAATCAAAATTTACCAGATAGTAACAGAATTTATGATTACAGTGAATA | |
| TGAAGATGTCACAAATGATGATTATGCCCAATTAGGAAATAATAATGACGTG | |
| AATATTAATTTTGGTAATATAGATTCACCATATATTATTAAAGTTATTAGTAAA | |
| TATGACCCTAATAAGGACGATTACACGACGATACAGCAAACTGTGACAATGC | |
| AAACGACTATAAATGAGTATACTGGTGAGTTTAGAACAGCATCCTATGATAA | |
| TACAATTGCTTTCTCTACAAGTTCAGGTCAAGGACAAGGTGACTTGCCTGCT | |
| GAAAAA | |
| Amino Acid Sequence | |
| MRGSHHHHHHGSEENTVQDVKDSNMDDELSDSNDQSSNEEKNDVINNSQSIN | (SEQ ID NO:2) |
| TDDDNQIKKEETNSNDAIENRSKDITQSTTNVDENEAIFLQKIPQDNTQLKEEV | |
| VKEPSSVESSNSSMDTAQQPSHTTINSEASIQTSDNEENSRVSDFANSKIIESNT | |
| ESNKEENTIEQPNKVREDSITSQPSSYKNIDEKISNQDELLNLPINEYENKVRPLS | |
| TTSAQPSSKRVTVNQLAAEQGSNVNHLIKVTDQSITEGYDDSDGIIKAHDAENLI | |
| YDVTFEVDDKVKSGDTMTVNIDKNTVPSDLTDSFAIPKIKDNSGEIIATGTYDNTN | |
| KQITYTFIDYVDKYENIKAHLKLTSYIDKSKVPNNNTKLDVEYKTALSSVNKTITV | |
| EYQKPNENRTANLQSMFTNIDTKNHTVEQTIYINPLRYSAKETNVNISGNGDEG | |
| STIIDDSTIIKVYKVGDNQNLPDSNRIYDYSEYEDVTNDDYAQLGNNNDVNINFG | |
| NIDSPYIIKVISKYDPNKDDYTTIQQTVTMQTTINEYTGEFRTASYDNTIAFSTSSG | |
| QGQGDLPPEK | |

SdrG N2N3 (273-597):

| | |
|---|---|
| Nucleotide Sequence | |
| ATGAGAGGATCGCATCACCATCACCATCACGGATCTCTGGTTCCTAGGGGA | (SEQ ID NO:3) |
| TCCGAACAAGGTTCGAATGTTAATCATTTAATTAAAGTTACTGATCAAAGTAT | |
| TACTGAAGGATATGATGATAGTGATGGTATTATTAAAGCACATGATGCTGAA | |
| AACTTAATCTATGATGTAACTTTTGAAGTAGATGATAAGGTGAAATCTGGTG | |
| ATACGATGACAGTGAATATAGATAAGAATACAGTTCCATCAGATTTAACCGA | |
| TAGTTTTGCAATACCAAAAATAAAAGATAATTCTGGAGAAATCATCGCTACAG | |
| GTACTTATGACAACACAAATAAACAAATTACCTACACTTTTACAGATTATGTA | |
| GATAAATATGAAAATATTAAAGCGCACCTTAAATTAACATCATACATTGATAA | |
| ATCAAAGGTTCCAAATAATAACACTAAGTTAGATGTAGAATATAAGACGGCC | |
| CTTTCATCAGTAAATAAAACAATTACGGTTGAATATCAAAAACCTAACGAAAA | |
| TCGGACTGCTAACCTTCAAAGTATGTTCACAAACATAGATACGAAAAACCAT | |
| ACAGTTGAGCAAACGATTTATATTAACCCTCTTCGTTATTCAGCCAAAGAAA | |
| CAAATGTAAATATTTCAGGGAATGGGGATGAAGGTTCAACAATTATCGACGA | |
| TAGTACAATCATTAAAGTTTATAAGGTTGGAGATAATCAAAATTTACCAGATA | |
| GTAACAGAATTTATGATTACAGTGAATATGAAGATGTCACAAATGATGATTAT | |
| GCCCAATTAGGAAATAATAATGACGTGAATATTAATTTTGGTAATATAGATTC | |
| ACCATATATTATTAAAGTTATTAGTAAATATGACCCTAATAAGGACGATTACA | |
| CGACGATACAGCAAACTGTGACAATGCAAACGACTATAAATGAGTATACTGG | |
| TGAGTTTAGAACAGCATCCTATGATAATACAATTGCTTTCTCTACAAGTTCAG | |
| GTCAAGGACAAGGTGACTTGCCTCCTGAAAAAT | |
| Amino Acid Sequence | |
| MRGSHHHHHHGSLVPRGSEQGSNVNHLIKVIDQSITEGYDDSDGIIKAHDAENL | (SEQ ID NO:4) |
| IYDVTFEVDDKVKSGDTMTVNIDKNTVPSDLTDSFAIPKIKDNSGEIIATGTYDNT | |
| NKQITYTFTDYVDKYENIKAHLKLTSYIDKSKVPNNNTKLDVEYKTALSSVNKTIT | |
| VEYQKPNENRTANLQSMFTNIDTKNHTVEQTIYINPLRYSAKETNVNISGNGDE | |
| GSTIIDDSTIIKVYKVGDNQNLPDSNRIYDYSEYEDVTNDDYAQLGNNNDVNINF | |
| GNIDSPYIIKVISKYDPNKDDYTTIQQTVTMQTTINEYTGEFRTASYDNTIAFSTSS | |
| GQGQGDLPPEK | |

SdrG TR2 (273-577):

| | |
|---|---|
| Nucleotide Sequence | |
| ATGAGAGGATCGCATCACCATCACCATCACGGATCCGAACAAGGTTCGAAT | (SEQ ID NO:5) |
| GTTAATCAT1TAATTAAAGTTACTGATCAAAGTATTACTGAAGGATATGATGA | |
| TAGTGATGGTATTATTAAAGCACATGATGCTGAAAACTTAATCTATGATGTAA | |
| CTTTTGAAGTAGATGATAAGGTGAAATCTGGTGATACGATGACAGTGAATAT | |
| AGATAAGAATACAGTTCCATCAGATTTAACCGATAGTTTTGCAATACCAAAAA | |
| TAAAAGATAATTCTGGAGAAATCATCGCTACAGGTACTTATGACAACACAAA | |
| TAAACAAATTACCTACACTTTTACAGATTATGTAGATAAATATGAAAATATTAA | |
| AGCGCACCTTAAATTAACATCATACATTGATAAATCAAAGGTTCCAAATAATA | |
| ACACTAAGTTAGATGTAGAATATAAGACGGCCTTTCATCAGTAAATAAAAC | |
| AATTACGGTTGAATATCAAAAACCTAACGAAAATCGGACTGCTAACCTTCAA | |
| AGTATGTTCACAAACATAGATACGAAAAACCATACAGTTGAGCAAACGATTT | |
| ATATTAACCCTCTTCGTTATTCAGCCAAAGAAACAAATGTAAATATTTCAGGG | |
| AATGGCGATGAAGGTTCAACAATTATCGAGGATAGTACAATCATTAAAGTTT | |
| ATAAGGTTGGAGATAATCAAAATTTACCAGATAGTAACAGAATTTATGATTAC | |
| AGTGAATATGAAGATGTCACAAATGATGATTATGCCCAATTAGGAAATAATA | |
| ATGACGTGAATATTAATTTTGGTAATATAGATTCACCATATATTATTAAAGTTA | |
| TTAGTAAATATGACCCTAATAAGGACGATTACACGACGATACAGCAAACTGT | |
| GACAATGCAAACGACTAIAAATGAGTATACTGGTGAGTTTAGAACAGCATCC | |
| TATTGA | |
| Amino Acid Sequence | |
| MRGSHHHHHHGSEQGSNVNHLIKVIDQSITEGYDDSDGIIKAHDAENLIYDVTF | (SEQ ID NO:6) |
| EVDDKVKSGDTMTVNIDKNTVPSDLTDSFAIPKIKDNSGEIIATGTYDNTNKQITY | |
| TFTDYVDKYENIKAHLKLTSYIDKSKVPNNNTKLDVEYKTALSSVNKTITVEYQKP | |
| NENRTANLQSMFTNIDTKNHTVEQTIYINPLRYSAKETNVNISGNGDEGSTIIDDS | |
| TIIKVYKVGDNQNLPDSNRIYDYSEYEDVTNDDYAQLGNNNDVNINFGNIDSPYII | |
| KVISKYDPNKDDYTTIQQTVTMQTTINEYTGEFRTASY | |

Protein Production and Purification

Using PCR, SdrGN1N2N3 (representing AA 50-597) or its subdomains such as SdrGN2N3 (representing AA 273-597) or its truncate TR2 (AA 273-577) were amplified from S. epidermidis K28 genomic DNA (from sequences described above) and subcloned into the E. coli expression vector PQE-30 (Qiagen), which allows for the expression of a recombinant fusion protein containing six histidine residues. This vector was subsequently transformed into the E. coli strain ATCC 55151, grown in a 15-liter fermentor to an optical density (OD₆₀₀) of 0.7 and induced with 0.2 mM isopropyl-1-beta-D galactoside (IPTG) for 4 hours. The cells were harvested using an AG Technologies hollow-fiber assembly (pore size of 0.45 □m) and the cell paste frozen at −80° C. Cells were lysed in 1×PBS (10 mL of buffer/1 g of cell paste) using 2 passes through the French Press @ 1100 psi. Lysed cells were spun down at 17,000 rpm for 30 minutes to remove cell debris. Supernatant was passed over a 5-mL HiTrap Chelating (Pharmacia) column charged with 0.1M NiCl₂. After loading, the column was washed with 5 column volumes of 10 mM Tris, pH 8.0, 100 mM NaCl (Buffer A). Protein was eluted using a 0-100% gradient of 10 mM Tris, pH 8.0, 100 mM NaCl, 200 mM imidazole (Buffer B) over 30 column volumes. SdrGN1N2N3, SdrGN2N3 or TR2 eluted at ˜13% Buffer B (˜26 mM imidazole). Absorbance at 280 nm was monitored. Fractions containing SdrGN1N2N3, SdrGN2N3 or TR2 were dialyzed in 1×PBS.

The protein was then put through an endotoxin removal protocol. Buffers used during this protocol were made endotoxin free by passing over a 5-mL Mono-Q sepharose (Pharmacia) column. Protein was divided evenly between 4×15 mL tubes. The volume of each tube was brought to 9 mL with Buffer A. 1 mL of 10% Triton X-114 was added to each tube and incubated with rotation for 1 hour at 4° C. Tubes were placed in a 37° C. water bath to separate phases. Tubes were spun down, at 2,000 rpm for 10 minutes and the upper aqueous phase from each tube was collected and the detergent extraction repeated. Aqueous phases from the 2nd extraction were combined and passed over a 5-mL IDA chelating (Sigma) column, charged with 0.1M NiCl₂ to remove remaining detergent. The column was washed with 9 column volumes of Buffer A before the protein was eluted with 3 column volumes of Buffer B. The eluant was passed over a 5-mL Detoxigel (Sigma) column and the flow-through collected and reapplied to the column. The flow-through from the second pass was collected and dialyzed in lx PBS. The purified product was analyzed for concentration, purity and endotoxin level before administration into the mice.

### Example 2

### Immunization Strategies for Monoclonal Antibody Production

With the goal of generating and characterizing monoclonal antibodies (mAbs), strategies were formulated to generate mAbs against SdrG that were of high affinity, able to interrupt or restrict the binding of fibrinogen to SdrG and demonstrate therapeutic efficacy in vivo. E. coli expressed and purified SdrG (N1N2N3, N2N3 or TR2) protein was used to generate a panel of murine monoclonal antibodies. Briefly, a group of Balb/C or SJL mice received a series of subcutaneous immunizations of 1-10 mg of protein in solution or mixed with adjuvant as described below in Table I:

TABLE I
Immunization Schemes

| | Day | Amount (μg) | Route | Adjuvant |
|---|---|---|---|---|
| RIMMS | | | | |
| Injection | | | | |
| #1 | 0 | 5 | Subcutaneous | FCA/RIBI |
| #2 | 2 | 1 | Subcutaneous | FCA/RIBI |
| #3 | 4 | 1 | Subcutaneous | FCA/RIBI |
| #4 | 7 | 1 | Subcutaneous | FCA/RIBI |
| #5 | 9 | 1 | Subcutaneous | FCA/RIBI |
| Conventional | | | | |
| Injection | | | | |
| Primary | 0 | 5 | Subcutaneous | FCA |
| Boost #1 | 14 | 1 | Intraperitoneal | RIBI |
| Boost #2 | 28 | 1 | Intraperitoneal | RIBI |
| Boost #3 | 42 | 1 | Intraperitoneal | RIBI |

At the time of sacrifice (RIMMS) or seven days after a boost (conventional) serum was collected and titered in ELISA assays against MSCRAMMs or on whole cells (S. epidermidis). Three days after the final boost, the spleens or lymph nodes were removed, teased into a single cell suspension and the lymphocytes harvested. The lymphocytes were then fused to a P3X63Ag8.653 myeloma cell line (ATCC #CRL-1580). Cell fusion, subsequent plating and feeding were performed according to the Production of Monoclonal Antibodies protocol from Current Protocols in Immunology (Chapter 2, Unit 2.).

### Example 3

### Screening and Selection of Anti-SdrG Monoclonal Antibodies

Any clones that were generated from the fusion were then screened for specific anti-SdrG antibody production using a standard ELISA assay. Positive clones were expanded and tested further for activity in a whole bacterial cell binding assay by flow cytometry and SdrG binding by Biacore analysis.

ELISA Analysis

Immulon 2-HB high-binding 96-well microtiter plates (Dynex) were coated with 1 μg/well of rClfA-(40-559) in 1×PBS, pH 7.4 and incubated for 2 hours at room temperature. All washing steps in ELISAs were performed three times with 1×PBS, 0.05% Tween-20 wash buffer. Plates were washed and blocked with a 1% BSA solution at room temperature for 1 hour before hybridoma supernatant samples were added to wells. Plates were incubated with samples and relevant controls such as media alone for one hour at room temperature, washed, and goat anti-mouse IgG-AP (Sigma) diluted 1:5000 in 1×PBS, 0.05% Tween-20, 0.1% BSA was used as a secondary reagent. Plates were developed by addition of 1 mg/ml solution of 4-nitrophenyl phosphate (pNPP) (Sigma), followed by incubation at 37° C. for 30 minutes. Absorbance was read at 405 nm using a SpectraMax 190 Plate Reader (Molecular Devices Corp.). Antibody supernatants that had an OD₄₀₅≧3 times above background (media alone, ˜0.1 OD) were considered positive.

Biacore Analysis

Throughout the analysis, the flow rate remained constant at 10 ml/min. Prior to the SdrGN1N2N3 or SdrGN2N3/TR2 injection, test antibody was adsorbed to the chip via RAM-Fc binding. At time 0, SdrG (N2N3, TR2 or N1N2N3) at a concentration of 30 mg/ml was injected over the chip for 3 min followed by 2 minutes of dissociation. This phase of the analysis measured the relative association and disassociation kinetics of the Mab/SdrG interaction.

Binding to Whole Bacteria

Bacterial samples (HB, 9142 or SdrG/lactococcus) were collected, washed and incubated with Mab or PBS alone (control) at a concentration of 2 mg/ml after blocking with rabbit IgG (50 mg/ml). Following incubation with antibody, bacterial cells were incubated with Goat-F(ab′)₂-Anti-Mouse-F(ab′)₂-FITC which served as the detection antibody. After antibody labeling, bacterial cells were aspirated through the FACScaliber flow cytometer to analyze fluorescence emission (excitation: 488, emission: 570). For each bacterial strain, 10,000 events were collected and measured.

TABLE II
SdrG Screening Summary

| | | | | # SdrG | | | | # Whole Cell |
|---|---|---|---|---|---|---|---|---|
| | | | # Growth | Positives | | # Biacore | | Binding Positives |
| | Immunization | | Positive | by ELISA | | Positives | | by Flow (% of |
| Fusion # | Protocol | Antigen | Wells | (% of total) | | (% of total) | | total) |
| Fusion 41 | RIMMS | SdrGN1N2N3 | 261 | 26 | (10%) | 14 | (5.4%) | 4 (1.5%) |
| Fusion 42 | RIMMS | SdrGN1N2N3 | 207 | 8 | (3.9%) | 4 | (1.9%) | 0 |
| Fusion 58 | RIMMS | SdrGN1N2N3 | 167 | 6 | (3.4%) | 6 | (3.4%) | 5 (3%) |
| Fusion 59 | RIMMS | SdrGN2N3 | 164 | 1 | (0.6%) | 1 | (0.6%) | 0 |
| Fusion 62 | Conventional | SdrGN2N3 | 1440 | 144 | (10%) | 74 | (5.1%) | 19 (1.3%) |
| Fusion 63 | Conventional | SdrGN2N3 | 1440 | 22 | (1.5%) | 9 | (0.6%) | 7 (0.5%) |
| Fusion 64 | Conventional | SdrGN1N2N3 | 2000 | 32 | (1.6%) | 8 | (0.4%) | 7 (0.4%) |
| Fusion 80 | Conventional | SdrGN2N3 | 1920 | 52 | (2.7%) | 11 | (0.6%) | ND |
| Fusion 81 | Conventional | SdrGN2N3 | 1920 | 32 | (1.8%) | 5 | (0.3%) | ND |
| Fusion 82 | Conventional | SdrGTR2 | 1440 | 7 | (0.5%) | 2 | (0.1%) | ND |
| Fusion 83 | Conventional | SdrGTR2 | 1440 | 21 | (1.5%) | 14 | (1%) | ND |

From the above analysis, SdrG positive hybridomas were generated in a frequency of 0.6-10% of the growth positive wells. Interestingly, very few SdrG ELISA positive hybridomas were also positive by Biacore analysis and whole cell bacterial binding by flow cytometry. Generally, Biacore negative, SdrG ELISA positive clones were negative in the whole cell binding flow cytometry assay. Examples of these observations are shown in Table III.

TABLE III
Representative Examples of Hybridoma Supernatants From Fusions in
Table II

| | Immunization | ELISA Data | Biacore | Flow Cytometric S. epi. |
|---|---|---|---|---|
| Fusion-Clone | Antigen | (SdrGN1N2N3) | Analysis | Staining |
| 41-19 | SdrGN1N2N3 | 0.276 | − | − |
| 41-75 | SdrGN1N2N3 | 0.831 | + | + |
| 41-129 | SdrGN1N2N3 | 1.195 | + | − |
| 41-206 | SdrGN1N2N3 | 0.780 | + | + |
| 41-211 | SdrGN1N2N3 | 0.731 | + | + |
| 42-31 | SdrGN1N2N3 | 0.537 | + | − |
| 42-76 | SdrGN1N2N3 | 0.266 | − | − |
| 59-59 | SdrGN2N3 | 0.459 | + | + |
| 62-27 | SdrGN2N3 | 0.555 | − | ND |
| 62-17 | SdrGN2N3 | 0.640 | + | − |
| 62-02 | SdrGN2N3 | 0.437 | + | − |
| 63-06 | SdrGN2N3 | 0.717 | + | + |
| 64-03 | SdrGN1N2N3 | 0.873 | + | + |
| 64-04 | SdrGN1N2N3 | 0.700 | + | + |
| 64-07 | SdrGN1N2N3 | 0.742 | + | + |
| 80-01 | SdrGN2N3 | 0.671 | − | + |
| 80-02 | SdrGN2N3 | 0.602 | + | + |
| 81-01 | SdrGN2N3 | 0.664 | + | + |
| 81-02 | SdrGN2N3 | 0.743 | + | + |
| 81-03 | SdrGN2N3 | 0.512 | + | + |
| 82-05 | SdrGTR2 | 0.892 | + | ND |
| 83-02 | SdrGTR2 | 0.753 | + | ND |
| 83-07 | SdrGTR2 | 0.731 | + | ND |
| 83-10 | SdrGTR3 | 0.654 | + | ND |
| 83-13 | SdrGTR2 | 0.671 | + | ND |
| 83-17 | SdrGTR2 | 0.678 | + | ND |
| 83-20 | SdrGTR2 | 0.631 | + | ND |
| 83-21 | SdrGTR2 | 0.564 | + | ND |

From this analysis, a very small subpopulation of growth positive hybridoma wells that were SdrG ELISA positive, SdrG Biacore positive and flow cytometry positive on Lactococcus/SdrG were single cell cloned and characterized as candidates for potential efficacy against S. epidermidis infection models. Table IV shows this preliminary characterization.

TABLE IV
Single Cell Cloned and Characterized SdrG Mabs.

| | | | | | Flow |
|---|---|---|---|---|---|
| | | | Biacore Analysis | | Cytometric |
| | Immunization | ELISA Data | Binding Phase | Dissociation Phase | SdrG |
| Fusion/clone | Antigen | (SdrGN1N2N3) | (RU) | (RU) | Staining |
| 41-75.3 | SdrGN1N2N3 | 0.831 | 218.7 | 173.3 | + |
| 41-206.4 | SdrGN1N2N3 | 0.899 | 83.3 | 66.4 | + |
| 41-211.3 | SdrGN1N2N3 | 0.739 | 80.4 | 64.2 | + |
| 59-59.4 | SdrGN2N3 | 0.459 | 19.0 | 8.6 | + |
| 62-23.4 | SdrGN1N2N3 | 0.517 | 103.0 | 87.2 | + |
| 62-37.10 | SdrGN2N3 | 0.425 | 22.5 | ND | + |
| 62-71.4 | SdrGN2N3 | 0.642 | 60.1 | ND | + |
| 63-02.6 | SdrGN2N3 | 0.673 | 27.3 | 28.2 | + |
| 63-03 | SdrGN2N3 | 0.621 | 47.4 | 37.1 | + |
| 63-08.4 | SdrGN2N3 | 0.639 | 24.6 | 24.3 | + |
| 64-03.6 | SdrGN1N2N3 | 0.562 | 29.5 | 30.1 | + |
| 64-04.3 | SdrGN1N2N3 | 0.818 | 11.6 | 13.4 | + |
| 64-07.3 | SdrGN1N2N3 | 0.846 | 20.5 | 20.7 | + |
| 80-01.21 | SdrGN2N3 | 0.671 | 3.7 | 1.2 | + |
| 80-02.4 | SdrGN2N3 | 0.602 | 490.7 | 453.6 | + |
| 81-01.12 | SdrGN2N3 | 0.664 | 553.3 | 487.0 | + |
| 81-02.1 | SdrGN2N3 | 0.743 | 821.2 | 767.8 | + |
| 81-03.5 | SdrGN2N3 | 0.512 | 425.4 | 289.8 | + |

### Example 4

### Binding Kinetics of Cloned Anti-SdrG Monoclonal Antibodies

Kinetic analysis was performed to demonstrate the diversity of the anti-SdrG mAbs chosen and characterized. As shown below the mAbs differ in there on-rate and off-rate as well as the overall affinity.

Biacore Kinetics

Kinetic analysis was performed on a Biacore 3000 using the Ligand capture method included in the software. A GAH-F(ab)₂ chip. The anti-SdrG mAbs were then passed over a GAM-F(ab)₂ chip, allowing binding to the Fc portion. Varying concentrations of the SdrG protein were then passed over the chip surface and data collected. Using the Biacore provided Evaluation software (Version 3.1), kₒₙ and k_{off} were measured and K_{A} and K_{D} were calculated.

TABLE V
Kinetic Analysis using the Biacore

| | | | kₐ | k_{d} | K_{A} | |
|---|---|---|---|---|---|---|
| | Run | | Association Rate; | Disassociation | Affinity | K_{D} Disassociation |
| Mab | # | Lot # | msec⁻¹ | Rate; sec⁻¹ | Constant; M⁻1 | Constant; M |
| 59-59 | R658 | IAA2E2122 | 3.42 × 10⁴ | 1.38 × 10⁻² | 2.48 × 10⁶ | 4.04 × 10⁻⁷ |
| 41-075 | R224 | Sup | 3.78 × 10⁵ | 2.72 × 10⁻³ | 1.39 × 10⁸ | 7.16 × 10⁻⁹ |
| 41-206 | R228 | Sup | 9.87 × 10⁴ | 2.53 × 10⁻³ | 3.97 × 10⁷ | 2.56 × 10⁻⁸ |
| 62-71 | R663 | IAA2C2049 | 6.07 × 10⁵ | 2.41 × 10⁻² | 2.52 × 10⁷ | 3.97 × 10⁻⁸ |
| 63-02 | R661 | IAA2B2030 | 3.28 × 10⁴ | 5.03 × 10⁻⁴ | 6.52 × 10⁷ | 1.53 × 10⁻⁸ |
| 64-03 | R660 | IAA2C2058 | 5.43 × 10⁴ | 2.84 × 10⁻⁴ | 1.91 × 10⁸ | 5.23 × 10⁻⁹ |
| 64-04 | R669 | IAA2J2260 | 9.94 × 10⁴ | 1.20 × 10⁻⁴ | 8.28 × 10⁸ | 1.21 × 10⁻⁹ |
| 64-07 | R670 | IAA2D2080 | 2.57 × 10⁴ | 5.58 × 10⁻⁴ | 4.60 × 10⁷ | 2.17 × 10⁻⁸ |

### Example 5

### Binding of Cloned Anti-SdrG Monoclonal Antibodies to Whole S. epidermidis Bacteria

To determine that the anti-SdrG mAbs generated and selected with recombinant SdrG cross-reacted with native SdrG expressed on Coagulase-negative Staph. bacteria flow cytometric analysis was used. In all cases the mAbs recognized the SdrG expressed on L. lactis, but varied in reactivity to HB and F40802.

Binding to Whole Bacteria

Bacterial samples (HB, F40802 or SdrG/lactococcus) were collected, washed and incubated with Mab or PBS alone (control) at a concentration of 2 mg/ml after blocking with rabbit IgG (50 mg/ml). Following incubation with antibody, bacterial cells were incubated with Goat-F_{(ab′)2}-Anti-Mouse-F_{(ab′)2}-FITC which served as the detection antibody. After antibody labeling, bacterial cells were aspirated through the FACScaliber flow cytometer to analyze fluorescence emission (excitation: 488, emission: 570). For each bacterial strain, 10,000 events were collected and measured. Units were determined by multiplying the percent of the gated positive events by the geometric mean of the stained population.

TABLE VI
Flow Cytometric Straining of Whole Coagulase-Negative
Stphylococcus Bacteria

| Purified Clone | | L. lactis SdrG | HB | F40802 |
|---|---|---|---|---|
| | 41-75.3 | 98,777 | 2,693 | 3,741 |
| | 41-206.4 | 121,237 | 1,766 | 2,032 |
| | 41-211.3 | 90,621 | 1,648 | 2,092 |
| | 59-59.4 | 29,976 | 6 | 1,509 |
| | 64-03.6 | 24,108 | 1,032 | 982 |
| | 64-04.3 | 23,892 | 1,362 | 1,015 |
| | 64-07.3 | 24,893 | 799 | 837 |
| | 80-01.21 | 2,665 | 16 | 25 |

### Example 6

### Inhibition of SdrG Binding to Fibrinogen

A number of the selected anti-SdrG mAbs of high affinity also displayed the ability to inhibit human fibrinogen or the p-fibrinogen peptide fragment binding to the SdrG MSCRAMM. This inhibition was characterized using a number of assays described below. This data suggests that is may be possible to inhibit the adhesive properties of the SdrG MSCRAMM to human fibrinogen.

Biacore Analysis—mAb Binding to SdrG Coupled with Inhibition of SdrG-Fibrinogen Binding

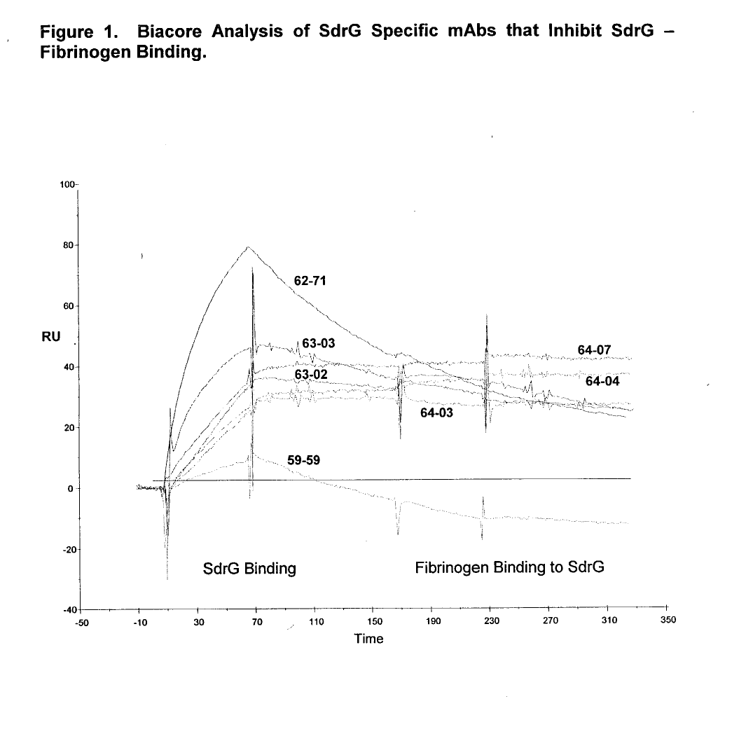

Throughout the analysis, the flow rate remained constant at 10 ml/min. Prior to the SdrGN1N2N3 or SdrGN2N3 injection, test antibody was adsorbed to the chip via RAM-Fc binding. At time 0, SdrG (N1N2 or N1N2N3) at a concentration of 30 mg/ml was injected over the chip for 3 min followed by 2 minutes of dissociation. This phase of the analysis measured the relative association and disassociation kinetics of the Mab/SdrG interaction. In the second phase of the analysis, the ability of the Mab bound SdrG to interact and bind fibrinogen was measured. Fibrinogen at a concentration of 100 mg/ml was injected over the chip and after 3 minutes a report point is taken. Examples of binding of some of the mAbs in accordance with the invention is shown in FIG. 1.

Biacore Analysis—mAb Inhibition of SdrG binding to the β-Fibrinogen Peptide Coupled to the Chip

The precise binding site for SdrG on the fibrinogen molecule has been localized to the N-terminal portion of the β-chain. For further analysis and characterization, we synthesized a peptide containing this site with the addition of an N-terminal Cysteine residue, the sequence being:

CNEEGFFSARGHRPLD (SEQ ID NO:7)

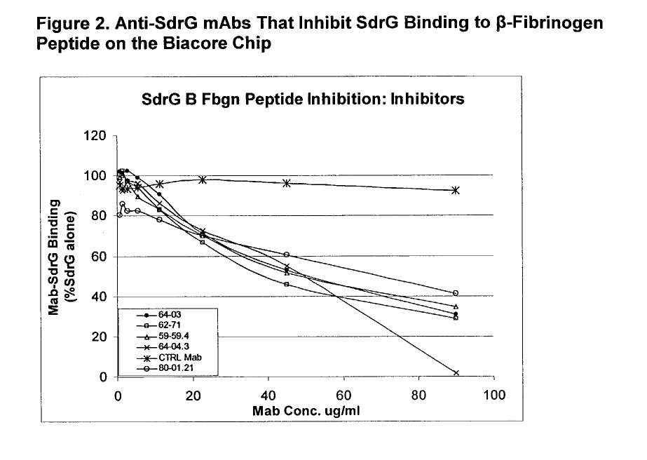

The β-Fibrinogen peptide is thiol-coupled to a research grade CM5 chip (Biacore) through the N-terminal cysteine according to the procedures detailed by Biacore. SdrG protein (30 μg/ml; full A-domain) is mixed with varying concentrations of mAb (90 μg/ml to 0.7 μg/ml) at a 1:1 ratio. The mixture was incubated at room temperature for 20 minutes and then passed over the β-Fibrinogen peptide chip and level of binding was measured. SdrG diluted 1:1 with buffer served as maximal SdrG binding, and incubation a non-SdrG mAb served as a negative control. Non-inhibitors should cause a large increase (above maximal SdrG binding) in Resonance Units (RUs) due to the large density of the SdrG/mAb complex binding to the peptide. Alternatively, inhibitors should reduce the level of binding below the maximal SdrG. Percent binding was determined as follows: Raw data, in terms of RUs, are divided by the SdrG control level multiplied by 100. Therefore SdrG with no mAb was always be 100% for a given experiment, allowing for comparisons between runs. Examples of mAbs in accordance with the invention showing the inhibition of SdrG Binding to the β-Fibrinogen peptide on the Biacore Chip is shown in FIG. 2.

ELISA-Based Protein Inhibition

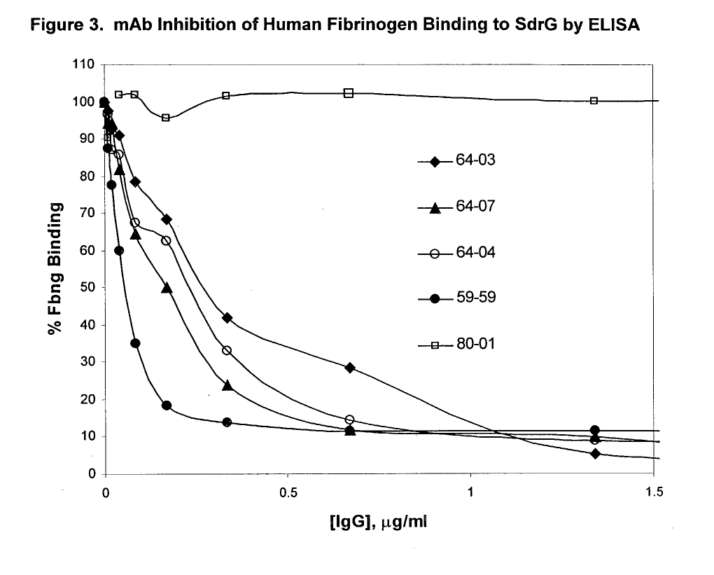

Immulon 2-HB high-binding 96-well plates were coated with 1 μg/ml SdrG (amino acids 50-597) or coagulase-negative staphylococcal protein (described in Example 7) in PBS and incubated 2 hours at room temperature. Plates were washed and blocked with 1% BSA solution for 1 hour, then washed and incubated with monoclonal antibody (either hybridoma supernatant or purified antibody) for 1 hour at room temperature. Following incubation with antibody, plates were either washed or left untreated, and 20 μg/ml human fibrinogen (Enzyme Research Lab, South Bend, Ind., USA) was added. Plates were incubated 1 hour at 37° C., washed, and goat anti-fibrinogen-HRP conjugate was added. Following incubation with conjugate, plates were washed and ABTS substrate was added. Plates then incubated 10 minutes at room temperature, the reaction was stopped with addition of 10% SDS, and absorbance was read at 405 nm. All data was analyzed using SOFTmax Pro v.3.1.2. software (Molecular Devices Corp., Sunnyvale, Calif., USA). MAbs in accordance with the invention exhibiting inhibition of Human Fibrinogen Binding to SdrG by ELISA are shown in FIG. 3.

### Example 7

### Cross-Reactivity of Anti-SdrG Monoclonal Antibodies to Other Bacterial Proteins

To assess potential cross-reactivity with other proteins found on coagulase-negative staphylococci, the protein described below, identified in gene bank as accession #Y17116, was cloned, expressed and purified using methods similar to the methods described in Example 1. Interestingly, considerable cross-reactivity with this protein was identified with a number of the anti-SdrG mAbs of the present invention which thus recognized this protein. One anti-SdrG mAb (59-59) with inhibitory activity against SdrG—fibrinogen binding however, did not cross-react and did not inhibit the binding of the protein described below with fibrinogen.

The full sequence of this protein (Gen Bank #Y17116), identified herein as SEQ ID NO:8 is as follows:

MINKKNNLLIKKKPIANKSNKYAIRKFTVGTASIVIGATLLFGLGHNEAK
AEENSVQDVKDSNTDDELSDSNDQSSDEEKNDVINNNQSINTDDNNQIIK
KEETNNYDGIEKRSEDRTESTTNVDENEATFLQKTPQDNTHLTEEEVKES
SSVESSNSSIDTAQQPSHTTINREESVQTSDNVEDSHVSDFANSKIKESN
TESGKEENTIEQPNKVKEDSTTSQPSGYTNIDEKISNQDELLNLPINEYE
NKARPLSTTSAQPSIKRVTVNQLAAEQGSNVNHLIKVTDQSITEGYDDSE
GVIKAHDAENLIYDVTFEVDDKVKSGDTMTVDIDKNTVPSDLTDSFTIPK
IKDNSGEIIATGTYDNKNKQITYTFTDYVDKYENIKAHLKLTSYIDKSKV
PNNNTKLDVEYKTALSSVNKTITVEYQRPNENRTANLQSMFTNIDTKNHT
VEQTIYINPLRYSAKETNVNISGNGDEGSTIIDDSTIIKVYKVGDNQNLP
DSNRIYDYSEYEDVTNDDYAQLGNNNDVNINFGNIDSPYIIKVISKYDPN
KDDYTTIQQTVTMQTTINEYTGEFRTASYDNTIAFSTSSGQGQGDLPPEK
TYKIGDYVWEDVDKDGIQNTNDNEKPLSNVLVTLTYPDGTSKSVRTDEDG
KYQFDGLKNGLTYKITFETPEGYTPTLKHSGTNPALDSEGNSVWVTINGQ
DDMTIDSGFYQTPKYSLGNYVWYDTNKDGIQGDDEKGISGVKVTLKDENG
NIISTTTTDENGKYQFDNLNSGNYIVHFDKPSGMTQITTDSGDDDEQDAD
GEEVHVTITDHDDFSIDNGYYDDESDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDSDSDSDSVSDSDSDSDSDSGSDSDSDSDSDSDNDSDLGNSSDKSTK
DKLPDTGANEDYGSKGTLLGTLFAGLGALLLGKRRKNRKNKN

The following amino acid sequence was also tested:

Amino Acid Sequence (60-608) (SEQ ID NO:9)

EENSVQDVKDSNTDDELSDSNDQSSDEEENDVINNNQSINSDDNNQINKK
EETNNNDGIEKSSEDRTESTTNVDENEATFLQKSPQDNTHLTEEEVKEPS
SVESSNSSIDTAQQPSHTTINREESVQTSDNVEDSHVSDFANSKIKESNT
ESGKEENTIEQPNKVKEDSTTSQPSGYTNIDEKISNQDELLNLPINEYEN
KARPLSTTSAQPSIKRVTVNQLAAEQGSNVNHLIKVTDQSITEGYDDSEG
VIKAHDAENLIYDVIFEVDDKVKSGDTMTVDIDKNTVPSDLTDSFTIPKI
KDNSGEIIATGTYDNKNKQITYTFTDYVDKYENIKAHLKLTSYIDKSKVP
NNNTKLDVEYKTALSSVNKTITVEYQRPNENRTANLQSMFTNIDTKNHTV
EQTIYINPLRYSAKETNVNISGNGDEGSTIIDDSTIIKVYKVGDNQNLPD
SNRIYDYSEYEDVTNDDYAQLGNNNDVNINFGNIDSPYIIKVISKYDPNK
DDYTTIQQTVTMQTTINEYTGEFRTASYDNTIAFSTSSGQGQGDLPPEK

In accordance with the invention, monoclonal and polyclonal antibodies can thus be raised which recognize the sequences set forth above.

Test results of ELISA-based mAb cross-reactivity are set forth in Table VII below:

TABLE VII
ELISA-Based mAb Cross-reactivity

| Purified Clone | SdrG N1N2N3 | SdrG N2N3 | Gen Bank #Y17116 |
|---|---|---|---|
| 41-75.3 | 0.90 | + | 0.81 |
| 41-206.4 | 0.78 | + | 0.76 |
| 41-211.3 | 0.73 | + | 0.65 |
| 59-59.4 | 0.59 | + | 0.11 |
| 64-03.6 | 0.87 | + | 0.80 |
| 64-04.3 | 0.70 | + | 0.68 |
| 64-07.3 | 0.74 | + | 0.67 |
| 80-01.21 | 0.67 | + | 0.67 |

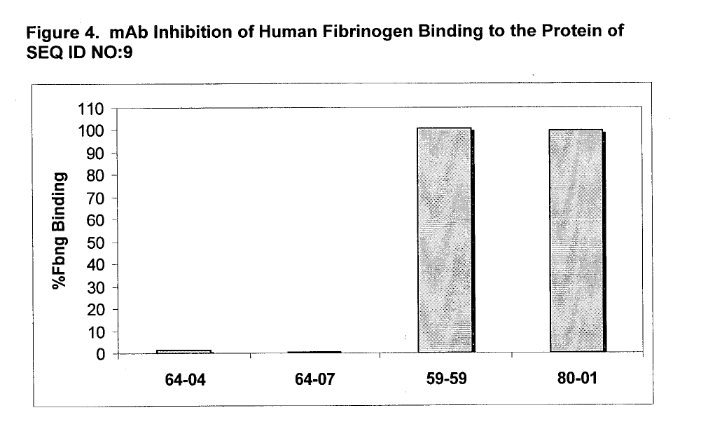

The results of the tests of mAb inhibition of human fibrinogen binding to Gen Bank protein of Accession No. Y17116 are shown in FIG. 4.

### Example 8

### In Vivo Based Therapeutic Activity

A number of anti-SdrG mAbs in accordance with the invention were tested for efficacy in in vivo animal models to demonstrate their potential utility as therapeutics.

Rodent Model of S. epidermidis Infection

Timed pregnant (13-15 day) Sprague-Dawley rats were purchased from Taconic Farms, (Germantown, N.Y.). 3-6 day old newborn rats were administered 0.35 mg of monoclonal antibody by a single intraperitoneal (IP) injection. Twenty hours following antibody administration, the newborn rats were challenged with an (IP) injection of 2×10⁸ CFU S. epidermidis strain HB. The survival of the animals was then followed for seven days. Kaplan-Meier analysis of survival curves was performed and significance was tested using a log rank test (Mantel-Haenszel Test). The test results are shown below:

Sex, Species, Number, Age, Weight and Source:

| Species | Strain | Sex | Number | Age | Weight | Source |
|---|---|---|---|---|---|---|
| Rat | Sprague | Male/ | 112 | 4-5 days | 9-16 | Charles |
| | Dawley | Female | | | grams | River |

Test Groups:

| | | TREATMENT | | | | CHALLENGE | | |
|---|---|---|---|---|---|---|---|---|
| Group | No. of | | Dose | Volume/Route/ | Time | | | Volume/ |
| # | Pups | Antibody | (mg) | Frequency | Point | Bacteria | Dose | Route |
| 1 | 10 | 41-211 | 0.35 mg | 0.20 ml/i.p./once | | S. Epidermidis | | 0.20 ml/i.p. |
| 2 | 10 | 41-075 | 0.35 mg | 0.20 ml/i.p./once | | | | |
| 3 | 10 | 41-206 | 0.35 mg | 0.20 ml/i.p./once | | | | |
| 4 | 10 | CRL-1771 | 0.35 mg | 0.20 ml/i.p./once | | | | |

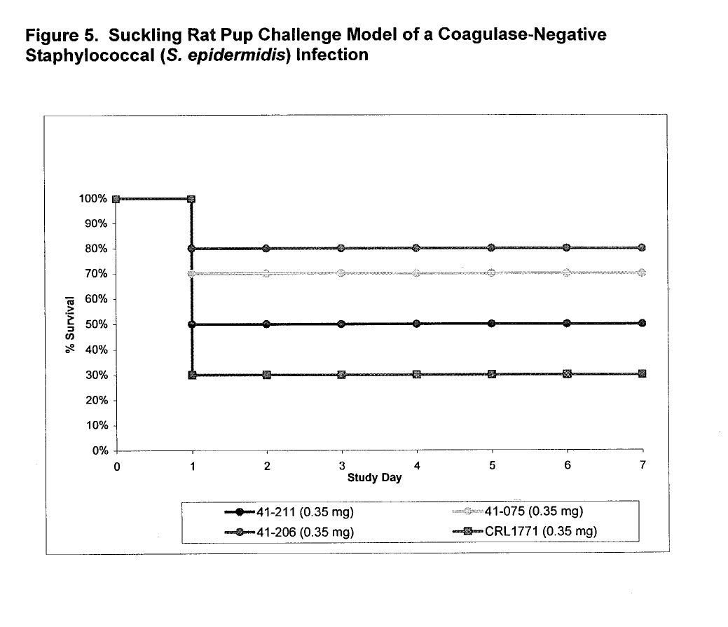

The results of the suckling Rat Pup Challenge Model of a Coagulase-Negative Staphylococcal (S. epidermidis) Infection are shown in FIG. 5.

Description of Antibody Test Reagents:

SdrG 41-211.3 Monoclonal Antibody, INH-M01023 (LN: IAA211454)

The 41-211.3 monoclonal antibody (IgG₁ subtype) was purified from serum free hybridoma culture medium using protein G affinity chromatography. The material was reported to be at a concentration of 10.4 mg/ml with an endotoxin concentration of <0.12 EU/mg of protein. The material was stored refrigerated at 4° C. On the day of injection, the material will be diluted to 1.75 mg/ml and 0.2 ml will be administered via an intraperitoneal injection to the appropriate group of animals. The final dose that will be administered will be 0.35 mg of IgG.

SdrG 41-075.3 Monoclonal Antibody, INH-M01024 (LN: IAA211447)

The 41-075.3 monoclonal antibody (IgG₁ subtype) was purified from serum free hybridoma culture medium using protein G affinity chromatography. The material was reported to be at a concentration of 7.6 mg/ml with an endotoxin concentration of <0.12 EU/mg of protein. The material was stored refrigerated at 4° C. On the day of injection, the material will be diluted to 1.75 mg/ml and 0.2 ml will be administered via an intraperitoneal injection to the appropriate group of animals. The final dose administered was 0.35 mg of IgG.

SdrG 41-206.4 Monoclonal Antibody, INH-M01025 (LN: IAA211448)

The 41-206.4 monoclonal antibody (IgG₁ subtype) was purified from serum free hybridoma culture medium using protein G affinity chromatography. The material was reported to be at a concentration of 8.9 mg/ml with an endotoxin concentration <0.12 EU/mg of protein. The material was stored refrigerated at 4° C. On the day of injection, the material will be diluted to 1.75 mg/ml and 0.2 ml will be administered via an intraperitoneal injection to the appropriate group of animals. The final dose administered was 0.35 mg of IgG.

Control CRL1771 Monoclonal Antibody, INH-M000029 (LN: IM2G1381)

The CRL 1771 monoclonal antibody (IgG₁ subtype) was purified from serum free hybridoma culture medium using protein G affinity chromatography. The material was reported to be at a concentration of 6.6 mg/ml with an endotoxin concentration of <3.0 EU/mg of protein. The material was stored refrigerated at 4° C. On the day of injection, the material will be diluted 1.75 mg/ml and 0.2 ml will be administered via an intraperitoneal injection to the appropriate group of animals. The final dose administered was 0.35 mg of IgG.

Rat Model of Central Venous Catheter (CVC) Associated Infection

8-9 week old male Sprague-Dawley rats were purchased from Charles River Laboratories (Raleigh, N.C.). A sterile polyethylene/silicon catheter (catheter body-polyethylene: 0.011″ id, 0.024″ od; catheter tip-silicon rubber: 0.012″ id, 0.025″ od) was surgically placed in the jugular vein and the catheter tip was advanced into the superior vena cava. The catheter remained in place and was kept patent throughout the study. Monoclonal antibodies were administered IV through the catheter at a dose of 20 mg/kg. 24 hours later, 5×10³ CFU of methicillin resistant S. epidermidis MRSE (Strain 899) were introduced via the catheter. Day 7 post-challenge, the animals were sacrificed and caudal vena cava blood, kidneys and catheter associated tissues were harvested. The MRSE colony forming units present in the tissue samples were measured by quantitative plating. Statistical analysis of the incidence of infection across groups was performed using Fisher's Exact Test. Statistical Analysis of quantitative differences in CFU between groups was performed using the Kruskal-Wallis Test with Dunn's multiple comparison post-test.

Description of Antibody Test Reagents:

SdrG 59-59.4 Monoclonal Antibody, INH-M02001 (LN: IAA2B2032)

The 41-211.3 monoclonal antibody (IgG₁ subtype) was purified from serum free hybridoma culture medium using protein G affinity chromatography. The material was reported to be at a concentration of 8.2 mg/ml with an endotoxin concentration of <0.12 EU/mg of protein. The material was stored refrigerated at 4° C. On the day of injection, the material was administered via the catheter for a final dose 20 mg/kg of IgG.

SdrG 64-03.6 Monoclonal Antibody, INH-M02008 (LN: IAA2C2058)

The 41-075.3 monoclonal antibody (IgG₁ subtype) was purified from serum free hybridoma culture medium using protein G affinity chromatography. The material was reported to be at a concentration of 11 mg/ml with an endotoxin concentration of <0.12 EU/mg of protein. The material was stored refrigerated at 4° C. On the day of injection, the material was administered via the catheter for a final dose 20 mg/kg of IgG.

Control CRL1771 Monoclonal Antibody, INH-M000029 (LN: IAA2G1381)

The CRL 1771 monoclonal antibody (IgG₁ subtype) was purified from serum free hybridoma culture medium using protein G affinity chromatography. The material was reported to be at a concentration of 6.6 mg/ml with an endotoxin concentration of <3.0 EU/mg of protein. The material was stored refrigerated at 4° C. On the day of injection, the material was administered via the catheter for a final dose 20 mg/kg of IgG.

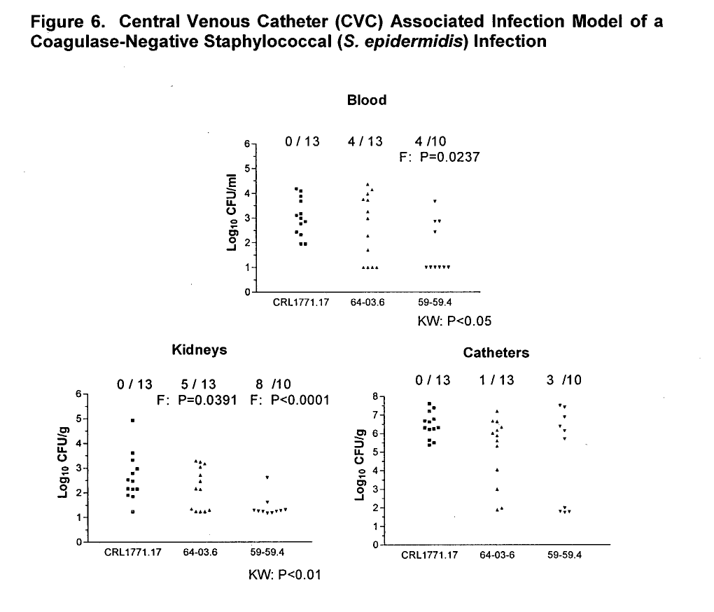

Test results showing the central venous catheter (CVC) associated infection model of a coagulase-negative Staphylococcal (S. epidermidis) Infection at Day 7 are shown in FIG. 6.

What is claimed is:

1. An isolated antibody that recognizes a protein from S. epidermidis selected from the group consisting of SdrG N1N2N3, SdrG N2N3 and SdrG TR2.

2. The antibody according to claim 1 wherein the antibody is a monoclonal antibody.

3. The monoclonal antibody according to claim 2 wherein the antibody is of a type selected from the group consisting of chimeric, murine, humanized and human monoclonal antibodies.

4. The monoclonal antibody according to claim 2 wherein the antibody is a single chain monoclonal antibody.

5. The antibody according to claim 1, wherein said antibody prevents a coagulase-negative staphylococcal infection in a human or animal.

6. The antibody according to claim 1, wherein said antibody inhibits binding of staphylococcal bacteria to fibrinogen.

7. The antibody according to claim 1, wherein said antibody is suitable for parenteral, oral, intranasal, subcutaneous, aerosolized or intravenous administration in a human or animal.

8. The antibody according to claim 1 wherein the antibody binds to the S. epidermidis SdrG protein.

9. The antibody according to claim 1 wherein the antibody recognizes an amino acid sequence selected from the group consisting of SEQ ID NO:2, SEQ ID NO:4 and SEQ ID NO:6.

10. The antibody according to claim 1 wherein the antibody recognizes an amino acid sequence encoded by a nucleic acid sequence selected from the group consisting of SEQ ID NO:1, SEQ ID NO:3 and SEQ ID NO:5 and degenerates thereof.

11. Isolated antisera containing an antibody according to claim 1.

12. A diagnostic kit comprising an antibody according to claim 1 and means for detecting binding by that antibody.

13. A diagnostic kit according to claim 12 wherein said means for detecting binding comprises a detectable label that is linked to said antibody.

14. A method of treating or preventing a coagulase-negative staphylococcal infection comprising administering to a human or animal patient an effective amount of an antibody according to claim 1.

15. A pharmaceutical composition for treating or preventing a coagulase-negative staphylococcal comprising an effective amount of the antibody of claim 1 and a pharmaceutically acceptable vehicle, carrier or excipient.

16. An isolated antibody that recognizes the protein sequence of SEQ ID NO:8.

17. The antibody according to claim 16 wherein the antibody is a monoclonal antibody.

18. A method of treating or preventing a coagulase-negative staphylococcal infection comprising administering to a human or animal patient an effective amount of an antibody according to claim 16.

19. A pharmaceutical composition for treating or preventing a coagulase-negative staphylococcal comprising an effective amount of the antibody of claim 16 and a pharmaceutically acceptable vehicle, carrier or excipient.

20. An isolated antibody that recognizes the amino acid sequence of SEQ ID NO:9.

21. The antibody according to claim 20 wherein the antibody is a monoclonal antibody.

22. A method of treating or preventing a coagulase-negative staphylococcal infection comprising administering to a human or animal patient an effective amount of an antibody according to claim 20.

23. A pharmaceutical composition for treating or preventing a coagulase-negative staphylococcal comprising an effective amount of the antibody of claim 20 and a pharmaceutically acceptable vehicle, carrier or excipient.

24. An isolated S. epidermidis protein selected from the group consisting of SdrG N1N2N3, SdrG N2N3 and SdrG TR2.

25. A method of eliciting an immunogenic reaction in a human or animal comprising administering to said human or animal an immunologically effective amount of an isolated protein according to claim 24.

26. A vaccine comprising an immunogenic amount of the isolated protein according to claim 24 and a pharmaceutically acceptable vehicle, carrier or excipient.

27. The isolated protein according to claim 24 wherein the protein has an amino acid sequence selected from the group consisting of SEQ ID NO:2, SEQ ID NO:4 and SEQ ID NO:6.

28. The isolated protein according to claim 24 wherein the protein is encoded by a nucleic acid sequence selected from the group consisting of SEQ ID NO:1, SEQ ID NO:3 and SEQ ID NO:5 and degenerates thereof.

29. An isolated nucleic acid sequence encoding a S. epidermidis protein selected from the group consisting of SdrG N1N2N3, SdrG N2N3 and SdrG TR2.

30. The isolated nucleic acid sequence according to claim 29 having a sequence selected from the group consisting of SEQ ID NO:1, SEQ ID NO:3 and SEQ ID NO:5, and degenerates thereof.
